(19) United States
(12) Patent Application Publication
(10) Pub. No.: US 2018/0271771 A1
(43) Pub. Date: Sep. 27, 2018

(54) POLYURETHANEUREA SOLUTIONS FOR COMPOSITIONS WITH ACTIVE INGREDIENTS OR FRAGRANCES

(71) Applicant: COVESTRO DEUTSCHLAND AG, Leverkusen (DE)

(72) Inventors: Sophie VIALA, Köln (DE); Sebastian DÖRR, Düsseldorf (DE); Paula Cristina Alves RODRIGUES, Düsseldorf (DE); Martin KRAUSE, Limberg (DE); Xiaohui XIONG, Shanghai (CN)

(21) Appl. No.: 15/541,886

(22) PCT Filed: Jan. 18, 2016
(86) PCT No.: PCT/EP2016/050898

§ 371 Date: Jul. 6, 2017

(30) Foreign Application Priority Data
Jan. 19, 2015 (CN) PCT/CN2015/070989

(51) Int. Cl. A61K 8/87; A61K 8/34; A61Q 15/00; A61K 8/26

(52) U.S. Cl. CPC A61K 8/87; A61K 8/34; A61K 2800/30; A61K 8/26; A61Q 15/00

(57) ABSTRACT

The present invention relates to a composition for application to the human and animal body or to plants, comprising at least one active ingredient and/or fragrance, comprising a polyurethane urea which is dissolved in a solvent or solvent mixture, the solvent consisting of one or more monohydroxy-functional alcohols, or a solvent mixture consisting of organic solvents, which contains ≥50% by weight, based on the total mass of the solvent mixture, of at least one monohydroxy-functional alcohol, is used. The invention additionally embraces the composition for treating the human or animal body, the use of the composition to coat substrates, the substrates not being the human or animal body, and the use of the composition for application to the human or animal body, the active ingredient not having a therapeutic effect.

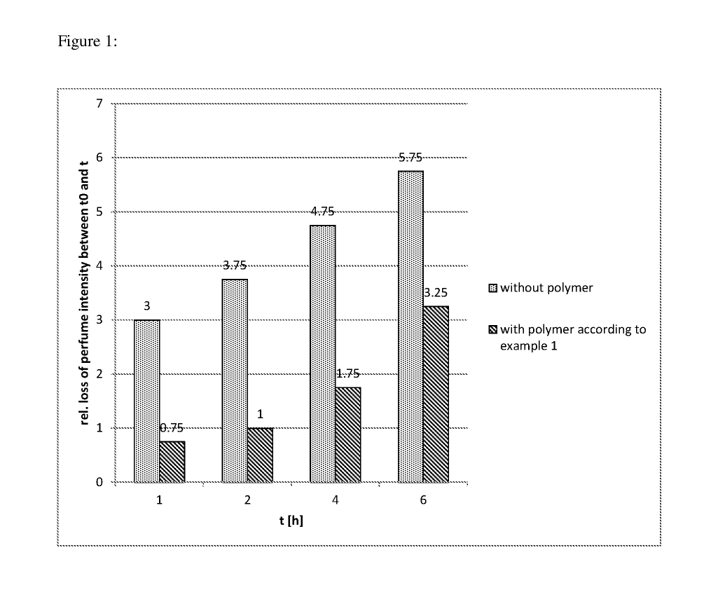

### CROSS-REFERENCE TO RELATED APPLICATIONS

This application is a national stage application (under 35 U.S.C. § 371) of PCT/EP2015/050898, filed Jan. 18, 2016, which claims benefit of Chinese national stage application PCT/CN2015/070989, filed Jan. 19, 2015, both of which are incorporated herein by reference in their entirety.

### FIELD OF THE INVENTION

The present invention relates to a composition comprising at least one active ingredient and fragrance and a specific polyurethane urea, and also to the composition for treating the human or animal body, to the use of the composition to coat substrates, the substrates not being the human or animal body, and to the use of the composition for application to the human or animal body, the active ingredient not having a therapeutic effect.

### BACKGROUND OF THE INVENTION

The controlled release or immobilization of active ingredients poses a great challenge for many applications. Areas of application for controlled-release formulations are found, for example, in agriculture, cosmetics and medicine. The goals that are important may differ according to the application, including, for example: controlled and/or delayed release of active ingredients, reduction in toxicity, reduced decomposition of the active ingredient, reduced volatility of the active ingredient, reduced odor of the formulation, or greater ease of handling.

Thus, for example, in the use of antiperspirants in deodorants, abrasion of the active ingredient frequently gives rise to the problem of yellowing of clothing. For this application, accordingly, it would be desirable to immobilize the active ingredient on the skin in such a way as to allow these adverse effects to be reduced or prevented.

A problem which often arises in products containing fragrances, such as perfumes, is that, while they have a very intensive odor directly after application to the skin, they nevertheless often lose their fragrancing effect rapidly. For these applications, accordingly, there is a continual demand for solutions which permit the fragrancing effect to be prolonged.

Disclosed in the art, for example, is the coating of medical articles and instruments with polyurethane solutions in order to achieve controlled release of active pharmacological ingredients. Suitable systems are, for example, in DE 10 2005 010 998 A1, WO 2009/115264 A1, WO 2010/025841 A1 and WO 2011/082946 A1. The polyurethane solutions described in the art, however, are not suitable for application on human or animal skin, since to prepare the solution it is always necessary to use solvents such as toluene, dimethylformamide or dimethylacetamide, which are unsuitable for application to the human and animal body or to plants, on account of their toxicological profile, which may be deleterious for the stated applications.

### SUMMARY OF THE INVENTION

The present invention therefore, provides a composition which is suitable for application to the human and animal body, and also to plants, which allows active ingredients and/or fragrances to be immobilized on the skin or plants in order, for example, to control their release or abrasion.

Compositions suitable for application to the human or animal body are more particularly those which do not exhibit any contraindication, such as suspicion or proof of cancerogenicity, mutagenicity or reproductive toxicity, for example.

These and other advantages and benefits of the present invention will be apparent from the Detailed Description of the Invention herein below.

### BRIEF DESCRIPTION OF THE FIGURE

The present invention will now be described for purposes of illustration and not limitation in conjunction with the FIGURE, wherein:

The FIGURE is a bar chart illustrating relative loss of perfume intensity over time.

### DETAILED DESCRIPTION OF THE INVENTION

The present invention will now be described for purposes of illustration and not limitation.

This invention provides a composition for application to the human and animal body or to plants, comprising at least one active ingredient and/or fragrance, characterized in that it further comprises a polyurethane urea which is present dissolved in a solvent or solvent mixture, the solvent consisting of one or more monohydroxy-functional alcohols, or a solvent mixture consisting of organic solvents, which contains ≥50% by weight, based on the total mass of the solvent mixture, of at least one monohydroxy-functional alcohol, is used.

Surprisingly it has been found that the compositions of the invention immobilize active ingredients and fragrances, allowing their release or abrasion to be controlled. It has been possible accordingly to achieve significant reduction in the abrasion of antiperspirants and to prolong the fragrancing effect of perfumes.

A further subject of the invention is a composition comprising at least one active ingredient and/or fragrance and at least one polyurethane urea which is present dissolved in a solvent or solvent mixture, the solvent consisting of one or more monohydroxy-functional alcohols, or a solvent mixture consisting of organic solvents, which contains ≥50% by weight, based on the total mass of the solvent mixture, of at least one monohydroxy-functional alcohol, is used, characterized in that the polyurethane urea is constructed from
a) at least one aliphatic, araliphatic and/or cycloaliphatic diisocyanate,
b) at least one polyetherpolyol having a number-average molecular weight Mn of ≥400 and ≤6000 g/mol and a hydroxyl functionality of ≥1.5 and ≤4,
c) at least one amino-functional compound which has at least two isocyanate-reactive amino groups,
d) optionally, at least one alcohol which has at least two hydroxyl groups and a molar mass of ≥60 and ≤399 g/mol,
e) optionally, at least one compound which has one group that is reactive towards isocyanate groups, and
f) optionally, ≤20% by weight, based on the total mass of the polyurethane urea, of at least one polyol having a number-average molecular weight Mn of ≥500 and ≤6000 g/mol and a hydroxyl functionality of ≥1.5 and ≤4 and being different from b).

The human and animal body for the purposes of this invention encompasses the entire outer surface of the body, in particular the skin and the keratinous materials present on the body, such as hairs and nails.

The dissolved polyurethane urea used in accordance with the invention, including the solvent or solvent mixture, is also referred to below as polyurethane urea solution.

In the context of the invention, dissolved means that, at 23° C., they are homogeneous, single-phase liquid mixtures of at least two substances which are clear. In the context of the present invention, clear means the turbidity values of the solution are ≤200 NTU (Nephelometric Turbidity Unit), preferably ≤50 NTU, particularly preferably ≤10 NTU and very particularly preferably ≤3 NTU. The turbidity values here are determined by means of a scattered-light measurement in the 90° angle (nephelometry) at a wavelength of the measurement radiation 860 nm in accordance with DIN EN ISO 7027, carried out at 23° C. using a laboratory turbidity measuring device model 2100AN from HACH LANGE GmbH, Berlin, Germany.

In the context of the invention, polyurethane ureas are polymeric compounds which have at least two, preferably at least three, urethane-group-containing repeat units

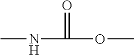

and moreover also urea-group-containing repeat units:

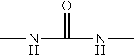

In a preferred embodiment of the invention, the polyurethane urea has no hydrophilizing groups, in particular no ionically or nonionically hydrophilizing groups.

In the context of the invention, ionically hydrophilizing groups are those which could be introduced into the polyurethane urea for example by means of suitable anionically or potentially anionically hydrophilizing compounds which have at least one isocyanate-reactive group such as a hydroxyl group or amino group, and at least one functionality such as e.g. —COO-M⁺, —SO₃-M⁺, —PO(O-M⁺)₂ where M⁺ is for example metal cation, H⁺, NH₄⁺, NHR₃⁺, where R can be in each case a C1-C12-alkyl radical, C5-C6-cycloalkyl radical and/or a C2-C4-hydroxyalkyl radical which, upon interaction with aqueous media, enters a pH-dependent dissociation equilibrium and in this way can be negatively or not charged. Suitable anionically or potentially anionically hydrophilizing compounds are mono- and dihydroxycarboxylic acids, mono- and dihydroxysulfonic acids, and also mono- and dihydroxyphosphonic acids and their salts. Examples of such anionic or potentially anionic hydrophilizing agents are dimethylolpropionic acid, dimethylolbutyric acid, hydroxypivalic acid, malic acid, citric acid, glycolic acid, lactic acid and the propoxylated adduct of 2-butenediol and NaHSO₃, as is described in DE-A 2 446 440, pages 5-9, formulae I-III.

In the context of the invention, nonionically hydrophilizing groups are those which could be introduced into the polyurethane urea, for example, by means of suitable nonionically hydrophilizing compounds, such as for example polyoxyalkylene ethers which contain at least one hydroxy or amino group. Examples are monohydroxy-functional polyalkylene oxide polyether alcohols having on statistical average 5 to 70, preferably 7 to 55, ethylene oxide units per molecule, as are accessible in a manner known by alkoxylation of suitable starter molecules (described e.g. in Ullmann's encyclopaedia of industrial chemistry, 4th edition, volume 19, Verlag Chemie, Weinheim pp. 31-38). These compounds are either pure polyethylene oxide ethers or mixed polyalkylene oxide ethers, in which case they can then however comprise at least 30 mol %, preferably at least 40 mol %, based on all of the alkylene oxide units present, of ethylene oxide units.

The polyurethane ureas used in the composition of the present invention are dissolved in a solvent or solvent mixture, and are thus used as polyurethane urea solutions and not as aqueous dispersions.

Preferably, the polyurethane urea used according to the invention comprises
a) at least one aliphatic, araliphatic and/or cycloaliphatic diisocyanate,
b) at least one polyetherpolyol with a number-average molecular weight Mn≥400 and ≤6000 g/mol and a hydroxyl functionality of ≥1.5 and ≤4,
c) at least one amino functional compound which has at least two isocyanate-reactive amino groups,
d) optionally, at least one alcohol which has at least two hydroxyl groups and a molar mass ≥60 and ≤399 g/mol,
e) optionally, at least one compound which has one group that is reactive towards isocyanate groups and
f) optionally, ≤20% by weight, based on the total mass of the polyurethane urea, of at least one polyol with a number-average molecular weight Mn≥500 and ≤6000 g/mol and a hydroxyl functionality of ≥1.5 and ≤4, which is different from b).

In the context of this application, the number-average molecular weight is always determined by gel permeation chromatography (GPC) in tetrahydrofuran at 23° C. The procedure here is in accordance with DIN 55672-1: “Gel permeation chromatography, Part 1—Tetrahydrofuran as eluent” (SECurity GPC System from PSS Polymer Service, flow rate 1.0 ml/min; columns: 2×PSS SDV linear M, 8×300 mm, 5 μm; RID detector). Here, polystyrene samples of known molar mass are used for the calibration. The calculation of the number-average molecular weight is assisted by software. Baseline points and evaluation limits are stipulated according to DIN 55672 Part 1.

Furthermore, the polyurethane urea preferably comprises ≥5 and ≤60% by weight of component a), ≥30 and ≤90% by weight of component b), ≥2 and ≤25% by weight of component c), ≥0 and ≤10% by weight of component d), ≥0 and ≤10% by weight of component e) and ≥0 and ≤20% by weight of component f), in each case based on the total mass of the polyurethane urea, where the components a) to f) add up to 100% by weight.

Furthermore, the polyurethane urea preferably comprises ≥10 and ≤40% by weight of component a), ≥55 and ≤85% by weight of component b), ≥5 and ≤20% by weight of component c), ≥0 and ≤3% by weight of component d), ≥0 and ≤3% by weight of component e) and ≥0 and ≤1% by weight of component f), in each case based on the total mass of the polyurethane urea, where the components a) to f) add up to 100% by weight.

Compounds suitable as component a) are, for example, 1,4-butylene diisocyanate, 1,5-pentamethylene diisocyanate (PDI), 1,6-hexamethylene diisocyanate (HDI), isophorone diisocyanate (IPDI), 2,2,4- and/or 2,4,4-trimethylhexamethylene diisocyanate, the isomeric bis(4,4′-isocyanatocyclohexyl)methanes or mixtures thereof with any desired isomer content (H12-MDI), 1,4-cyclohexylene diisocyanate, 4-isocyanatomethyl-1,8-octane diisocyanate (nonane triisocyanate), 1,3- and/or 1,4-bis(2-isocyanatoprop-2-yl)benzene (TMXDI), 1,3-bis(isocyanatomethyl)benzene (XDI), and alkyl 2,6-diisocyanatohexanoates (lysine diisocyanates) with C1-C8-alkyl groups.

Besides the polyisocyanates specified above, modified diisocyanates or triisocyanates with isocyanurate, urethane, allophanate, biuret, iminooxadiazinedione and/or oxadiazinetrione structure can also be co-used proportionately.

Preferably, they are polyisocyanates or polyisocyanate mixtures of the aforementioned type with an average NCO functionality of ≥2 and ≤4, preferably ≥2 and ≤2.6 and particularly preferably ≥2 and ≤2.4.

Preferably, the component a) is selected from aliphatic, araliphatic and/or cycloaliphatic diisocyanates which have at least one isocyanate group which is bonded to a secondary and/or tertiary carbon atom.

Particularly preferably, the component a) is selected from IPDI and/or H12-MDI.

Furthermore, preferably, no aromatic polyisocyanates are used for producing the polyurethane urea.

Component a) is preferably used in amounts of ≥5 and ≤60% by weight, particularly preferably ≥10 and ≤40% by weight and very particularly preferably from ≥15 and ≤35% by weight, based on the total weight of the polyurethane urea.

Component b) consists of one or more polyetherpolyols with a number-average molecular weight Mn≥400 and ≤6000 g/mol and a hydroxyl functionality of ≥1.5 and ≤4, preferably with a number-average molecular weight Mn≥500 and ≤2500 g/mol and a hydroxyl functionality of ≥1.9 and ≤3 and particularly preferably with a number-average molecular weight Mn≥1000 and ≤2000 g/mol and a hydroxyl functionality of ≥1.9 and ≤2.1.

Suitable polyetherpolyols of component b) are for example the poly(tetramethylene glycol) polyetherpolyols known in polyurethane chemistry, as are available by polymerization of tetrahydrofuran by means of cationic ring-opening.

Likewise, suitable polyetherpolyols are the addition products of styrene oxide, ethylene oxide, propylene oxide, butylene oxide and/or epichlorohydrin onto di- or polyfunctional starter molecules. Thus, in particular polyalkylene glycols, such as polyethylene, polypropylene and/or polybutylene glycols, can be used, especially with the aforementioned preferred molecular weights. The polyetherpolyols here preferably have a fraction of groups obtained from ethylene oxide of <50% by weight, preferably <30% by weight. In a preferred embodiment, no ethylene oxide is used to prepare compound b).

Suitable starter molecules that can be used are all compounds known in the art, such as for example water, butyl diglycol, glycerol, diethylene glycol, trimethyolpropane, propylene glycol, sorbitol, ethylenediamine, triethanolamine, 1,4-butanediol.

Preferably, component b) is selected from polypropylene glycols and/or poly(tetramethylene glycol) polyetherpolyols, particularly preferably selected from poly(tetramethylene glycol) polyetherpolyols.

In a preferred embodiment of the invention, component b) is one or more poly(tetramethylene glycol) polyetherpolyols with a with a number-average molecular weight Mn≥500 and ≤2500 g/mol and a hydroxyl functionality of ≥1.9 and ≤2.1.

In a particularly preferred embodiment, component b) is a mixture of poly(tetramethylene glycol) polyetherpolyols I with a number-average molecular weight Mₙ of ≥400 and ≤1500 g/mol, particularly preferably of ≥600 and ≤1200 g/mol, very particularly preferably of 1000 g/mol and poly(tetramethylene glycol) polyetherpolyols II with a number-average molecular weight Mₙ of ≥1500 and ≤8000 g/mol, particularly preferably of ≥1800 and ≤3000 g/mol, very particularly preferably of 2000 g/mol.

The weight ratio of the poly(tetramethylene glycol) polyetherpolyols I to the poly(tetramethylene glycol) polyetherpolyols II is preferably in the range from ≥0.1 and ≤10, particularly preferably in the range from ≥0.2 and ≤8, very particularly preferably in the range from ≥1 and ≤6.

Component b) is preferably used in amounts of ≥30 and ≤90% by weight, particularly preferably ≥50 and ≤85% by weight, very particularly preferably ≥55 and ≤75% by weight, based on the total weight of the polyurethane urea.

Component c) is one or more amino-functional compounds which have at least two isocyanate-reactive amino groups.

Of suitability as component c) are for example di- or polyamines such as 1,2-ethylenediamine, 1,2- and 1,3-diaminopropane, 1,4-diaminobutane, 1,6-diaminohexane, 2,2,4- and 2,4,4-trimethylhexamethylenediamine, 2-methylpentamethylenediamine, diethylenetriamine, triaminononane, 1,3- and 1,4-xylylenediamine, α,α,α′,α′-tetramethyl-1,3- and -1,4-xylylenediamine and 4,4′-diaminodicyclohexylmethane (H12-MDA), isophoronediamine (IPDA) and/or 1,2-dimethylethylenediamine.

Component c) is preferably selected from ethylenediamine, IPDA and/or H12-MDA, particularly preferably from isophoronediamine and/or H12-MDA and component c) is very particularly preferably H12-MDA.

The compounds of component c) preferably contain no hydrophilizing groups, in particular, no ionically hydrophilizing groups.

In a particularly preferred embodiment of the invention, component c) is selected from amines which have at least two isocyanate-reactive amino groups which are bonded to primary and/or secondary carbon atoms.

Component c) is furthermore preferably selected from symmetrically structured diamines.

Component c) is very particularly preferably selected from symmetrical diamines which have at least two amino groups which are bonded to primary and/or secondary carbon atoms, component c) being particularly preferably H12-MDA.

Component c) is preferably used in amounts of ≥2 and ≤25% by weight, particularly preferably ≥5 and ≤20% by weight and very particularly preferably ≥9 and ≤16% by weight, based on the total weight of the polyurethane urea.

In a preferred embodiment of the invention, either component a) is H12-MDI or component c) is H12-MDA or component a) is H12-MDI and component c) is H12-MDA.

Optionally, the polyurethane urea moreover comprises component d), one or more alcohols which have at least two hydroxyl groups and a molar mass of ≥60 and ≤399 g/mol, such as for example polyols of the stated molar mass range with up to 20 carbon atoms, such as ethylene glycol, diethylene glycol, triethylene glycol, 1,2-propanediol, 1,3-propanediol, 1,4-butanediol, 1,3-butylene glycol, cyclohexanediol, 1,4-cyclohexanedimethanol, 1,6-hexanediol, neopentyl glycol, hydroquinone dihydroxyethyl ether, bisphenol A (2,2-bis(4-hydroxy-phenyl)propane), hydrogenated bisphenol A, (2,2-bis(4-hydroxy-cyclohexyl)propane), trimethylolpropane, glycerol, pentaerythritol.

Component d) is preferably used in amounts of ≥0 and ≤10% by weight, particularly preferably ≥0 and ≤3% by weight, based on the total weight of the polyurethane urea, and is very particularly preferably not used at all.

Furthermore, the polyurethane urea can comprise component e), one or more compounds which have one group that is reactive towards isocyanate groups, in particular, compounds which have an amino or hydroxyl group. Suitable compounds of components e) are for example methylamine, ethylamine, propylamine, butylamine, octylamine, laurylamine, stearylamine, isononyloxypropylamine, dimethylamine, diethylamine, dipropylamine, dibutylamine, N-methyl-aminopropylamine, diethyl(methyl)aminopropylamine, morpholine, piperidine, methanol, ethanol, isopropanol, n-propanol, n-butanol, ethylene glycol monobutyl ether, diethylene glycol monomethyl ether, diethylene glycol monobutyl ether, propylene glycol monomethyl ether, dipropylene glycol monomethyl ether, tripropylene glycol monomethyl ether, dipropylene glycol monopropyl ether, propylene glycol monobutyl ether, dipropylene glycol monobutyl ether, tripropylene glycol monobutyl ether, 2-ethylhexanol, 1-octanol, 1-dodecanol, 1-hexadecanol.

Component e) preferably comprises no monofunctional polyetherpolyols which have a fraction of groups obtained from ethylene oxide of >30% by weight, preferably >50% by weight.

The monohydroxy-functional alcohol used as solvent for the polyurethane urea can likewise serve as structural component e) for the polyurethane urea.

Component e) is preferably used in amounts of ≥0 and ≤10% by weight, particularly preferably ≥0 and ≤3% by weight, based on the total weight of the polyurethane urea, and is very particularly preferably not used at all, in which case the monohydroxy-functional alcohol used as solvent for the polyurethane urea is not taken into consideration here as component e).

The monohydroxy-functional alcohol which serves as solvent for the polyurethane urea comprises preferably ≥0 and ≤5% by weight, particularly preferably ≥0.01 and ≤3% by weight and very particularly preferably ≥0.01 and ≤2% by weight, of the total mass of the polyurethane urea.

Optionally, the polyurethane urea can also comprise the component f), one or more polyols with a number-average molecular weight Mn≥500 and ≤6000 g/mol and a hydroxyl functionality of ≥1.5 and ≤4, where the polyols are different from b).

Component f) is preferably used in amounts of ≥0 and ≤20% by weight, particularly preferably ≥0 and ≤10% by weight, based on the total weight of the polyurethane urea, and is very particularly preferably not used at all.

Preferably, the polyols of component f) have a number-average molecular weight Mn≥1000 and ≤3000 g/mol and a hydroxyl functionality of ≥1.8 and ≤3.

Polyols suitable as component f) are the polyesterpolyols, polyacrylatepolyols, polyurethane-polyols, polycarbonatepolyols, polyesterpolyacrylatepolyols, polyurethane-polyacrylatepolyols, polyurethane-polyesterpolyols, polyurethane-polyetherpolyols, polyurethane-polycarbonatepolyols, polyetherpolycarbonate-polyols and/or polyesterpolycarbonatepolyols, in particular polyesterpolyols and/or polycarbonatepolyols, known in polyurethane coating technology.

Polyesterpolyols are for example the polycondensates of di- and optionally tri-, and tetraols and di- and optionally tri- and tetracarboxylic acids or hydroxycarboxylic acids or lactones. Instead of the free polycarboxylic acids, the corresponding polycarboxylic anhydrides or corresponding polycarboxylic acid esters of lower alcohols can also be used for preparing the polyesters.

Examples of diols suitable for this purpose are ethylene glycol, butylene glycol, diethylene glycol, triethylene glycol, polyalkylene glycols such as polyethylene glycol, also 1,2-propanediol, 1,3-propanediol, butanediol(1,3), butanediol(1,4), hexanediol(1,6) and isomers, neopentyl glycol or hydroxypivalic acid neopentyl glycol ester, with hexanediol(1,6) and isomers, neopentyl glycol and hydroxypivalic acid neopentyl glycol ester being preferred. In addition, it is also possible to use polyols such as trimethylolpropane, glycerol, erythritol, pentaerythritol, trimethylolbenzene or trishydroxyethyl isocyanurate.

Dicarboxylic acids which can be used are phthalic acid, isophthalic acid, terephthalic acid, tetrahydrophthalic acid, hexahydrophthalic acid, cyclohexanedicarboxylic acid, adipic acid, azelaic acid, sebacic acid, glutaric acid, tetrachlorophthalic acid, maleic acid, fumaric acid, itaconic acid, malonic acid, suberic acid, 2-methylsuccinic acid, 3,3-diethylglutaric acid and/or 2,2-dimethylsuccinic acid. As acid source, it may also be possible to use the corresponding anhydrides.

If the average hydroxyl functionality of the polyol to be esterified is greater than 2, then monocarboxylic acids, such as benzoic acid and hexanecarboxylic acid, can additionally also be co-used.

Preferred acids are aliphatic or aromatic acids of the aforementioned type. Particular preference is given to adipic acid, isophthalic acid and optionally trimellitic acid, very particularly preferably adipic acid.

Hydroxycarboxylic acids which can be co-used as reaction participants in the preparation of a polyesterpolyol with terminal hydroxyl groups are, for example, hydroxycaproic acid, hydroxybutyric acid, hydroxydecanoic acid, hydroxystearic acid and the like. Suitable lactones are caprolactone, butyrolactone and homologues. Preference is given to caprolactone.

In component f), it is also possible for polycarbonates having hydroxyl groups, preferably polycarbonate diols, with number-average molecular weights Mn of from 400 to 8000 g/mol, preferably from 600 to 3000 g/mol, to be used. These are obtainable by reaction of carbonic acid derivatives, such as diphenyl carbonate, dimethyl carbonate or phosgene, with polyols, preferably diols.

Examples of such diols are ethylene glycol, 1,2- and 1,3-propanediol, 1,3- and 1,4-butanediol, 1,6-hexanediol, 1,8-octanediol, neopentyl glycol, 1,4-bis-hydroxymethylcyclohexane, 2-methyl-1,3-propanediol, 2,2,4-trimethy-lpentanediol-1,3, dipropylene glycol, polypropylene glycols, dibutylene glycol, polybutylene glycols, bisphenol A and lactone-modified diols of the aforementioned type. The polycarbonates having hydroxyl groups are preferably linear in structure.

In a preferred embodiment of the invention, the polyurethane urea used according to the invention comprises
a) at least one aliphatic, araliphatic and/or cycloaliphatic diisocyanate which has at least one isocyanate group which is bonded to a secondary and/or tertiary carbon atom,
b) at least one polyetherpolyol with a number-average molecular weight Mn≥500 and ≤2500 g/mol and a hydroxyl functionality of ≥1.9 and ≤3,
c) at least one amino-functional compound which has at least two (isocyanate-reactive) amino groups and is selected from ethylenediamine, IPDA and/or H12-MDA,
d) optionally, at least one alcohol which has at least two hydroxyl groups and a molar mass ≥60 and ≤399 g/mol,
e) optionally, at least one compound which has one group that is reactive towards isocyanate groups and
f) optionally, ≤20% by weight, based on the total mass of the polyurethane urea, of at least one polyol with a number-average molecular weight Mn≥500 and ≤6000 g/mol and a hydroxyl functionality of ≥1.5 and ≤4, which is different from b).

Furthermore preferably, the polyurethane urea in this aforementioned embodiment comprises ≥5 and ≤60% by weight of component a), ≥30 and ≤90% by weight of component b), ≥2 and ≤25% by weight of component c), ≥0 and ≤10% by weight of component d), ≥0 and ≤10% by weight of component e) and ≥0 and ≤20% by weight of component f), in each case based on the total mass of the polyurethane urea, where the components a) to f) add up to 100% by weight.

Particularly preferably, the polyurethane urea in this aforementioned embodiment comprises ≥10 and ≤40% by weight of component a), ≥55 and ≤85% by weight of component b), ≥5 and ≤20% by weight of component c), ≥0 and ≤3% by weight of component d), ≥0 and ≤3% by weight of component e) and ≥0 and ≤1% by weight of component f), in each case based on the total mass of the polyurethane urea, where the components a) to f) add up to 100% by weight.

In a particularly preferred embodiment of the invention, the polyurethane urea used according to the invention comprises
a) at least one diisocyanate selected from IPDI and/or H12-MDI,
b) at least one polyetherpolyol with a number-average molecular weight Mn≥500 and ≤2500 g/mol and a hydroxyl functionality of ≥1.9 and ≤3, selected from polypropylene glycols and/or poly(tetramethylene glycol) polyetherpolyols,
c) at least one amino-functional compound selected from IPDA and/or H12-MDA,
d) optionally, at least one alcohol which has at least two hydroxyl groups and a molar mass ≥60 and ≤399 g/mol,
e) optionally, at least one compound which has one group which is reactive towards isocyanate groups and
f) optionally, ≤20% by weight, based on the total mass of the polyurethane urea, of at least one polyol with a number-average molecular weight Mn≥500 and ≤6000 g/mol and a hydroxyl functionality of ≥1.5 and ≤4, which is different from b).

Furthermore preferably, the polyurethane urea in this aforementioned embodiment comprises ≥5 and ≤60% by weight of component a), ≥30 and ≤90% by weight of component b), ≥2 and ≤25% by weight of component c), ≥0 and ≤10% by weight of component d), ≥0 and ≤10% by weight of component e) and ≥0 and ≤20% by weight of component f), in each case based on the total mass of the polyurethane urea, where the components a) to f) add up to 100% by weight.

Particularly preferably, the polyurethane urea in this aforementioned embodiment comprises ≥10 and ≤40% by weight of component a), ≥55 and ≤85% by weight of component b), ≥5 and ≤20% by weight of component c), ≥0 and ≤3% by weight of component d), ≥0 and ≤3% by weight of component e) and ≥0 and ≤1% by weight of component f), in each case based on the total mass of the polyurethane urea, where the components a) to f) add up to 100% by weight.

Preferably, the polyurethane urea comprises components a) to c) and optionally d) to f), particularly preferably consists of the components a) to c).

The polyurethane urea advantageously has a number-average molecular weight Mn≥2000 and ≤50 000 g/mol, particularly advantageously ≥3000 and ≤30 000 g/mol.

The polyurethane urea is preferably prepared by reacting the components a) and b), and optionally d) and f) in a first step to give an NCO-terminated prepolymer, which is then reacted in a subsequent step with the component c) and optionally the components d) and e).

For the preparation of the polyurethane ureas, the components a) and b) and also optionally d) and f) for the preparation of an NCO-terminated prepolymer are completely or partially introduced, optionally diluted with a solvent that is inert towards isocyanate groups and heated to temperatures in the range from 50 to 120° C. To increase the rate of the isocyanate addition reaction, the catalysts known in polyurethane chemistry can be used. In one preferred variant, however, processing takes place without the addition of urethanization catalysts.

Then, the constituents of a) and b) and also optionally d) and f) possibly still not added at the start of the reaction are metered in.

During the preparation of the NCO-terminated prepolymers of components a) and b) and also optionally d) and f), the quantitative ratio of isocyanate groups to isocyanate-reactive groups is generally ≥1.05 and ≤3.5, preferably ≥1.1 and ≤3.0, particularly preferably ≥1.1 and ≤2.5.

Isocyanate-reactive groups are to be understood as meaning all groups that are reactive towards isocyanate groups, such as for example primary and secondary amino groups, hydroxy groups or thiol groups.

The reaction of components a) and b) and also optionally d) and f) to give the prepolymer takes place partially or completely, but preferably completely.

Polyurethane prepolymers which contain free isocyanate groups are thus obtained in bulk or in solution.

Preferably, the NCO-terminated prepolymer is prepared exclusively from components a) and b).

Then, preferably in a further process step, if this has still not taken place or has taken place only partially, the resulting prepolymer is dissolved with the help of one or more organic solvents. The solvent used here is preferably likewise a solvent or solvent mixture, where the solvent consists exclusively of one or more monohydroxy-functional alcohols, or a solvent mixture consisting of exclusively organic solvents which comprises ≥50% by weight, based on the total mass of the solvent mixture, of at least one monohydroxy-functional alcohol, is used. For the solvent and the solvent mixture, the preferred embodiments below relating to solvent or solvent mixture in which the polyurethane urea is dissolved are likewise applicable. The solvent or solvent mixture here can also be different from the solvent or solvent mixture in which the polyurethane urea is later dissolved as end product. Preferably, the solvent or solvent mixture is identical to the solvent or solvent mixture in which the polyurethane urea is later dissolved as end product.

Preferably, the solvent used in the preparation consists of one or more monohydroxy-functional alcohols.

The ratio of solvent to prepolymer is here preferably ≥1:10 and ≤5:1, particularly preferably ≥1:2 and ≤2:1 parts by weight.

The prepolymer is preferably cooled prior to the dissolution to temperatures of −20 to 60° C., preferably 0 to 50° C. and particularly preferably from 15 to 40° C.

In a further step, which optionally follows the dissolution of the NCO-terminated prepolymer, the NCO-terminated prepolymer obtained in the first step is then preferably reacted completely or partially with component c) and also optionally components d) and e). This reaction is generally referred to as chain extension, or in the case of component e) as chain termination.

Preferably here, the NCO-terminated prepolymer is initially introduced and component c) and also optionally d) and e) are metered in. Preferably, firstly a partial reaction of the NCO groups of the prepolymer with component c) and optionally d) takes place, and then the chain termination by reaction of the remaining NCO groups with component e). Components c) and optionally e) can also be added here in stages in several steps, in particular in two steps.

Component c) and also optionally d) and e) are preferably used dissolved in one or more organic solvents. The solvent used here is preferably likewise a solvent or solvent mixture, where the solvent consists of one or more monohydroxy-functional alcohols, or a solvent mixture consisting of organic solvents which comprises ≥50% by weight, based on the total mass of the solvent mixture, of at least one monohydroxy-functional alcohol, is used. For the solvent and the solvent mixture, the preferred embodiments below relating to the solvent or solvent mixture in which the polyurethane urea is dissolved are likewise applicable.

The solvent or solvent mixture here can also be different from the solvent or solvent mixture in which the polyurethane urea is later dissolved as end product. Preferably, the solvent or solvent mixture is identical to the solvent or solvent mixture in which the polyurethane urea is later dissolved as end product. Preferably, the solvent for component c) used in the preparation consists of one or more monohydroxy-functional alcohols.

If solvents are used as diluents, then the diluent content in components c) and also optionally d) and e) used in the chain extension is preferably 1 to 95% by weight, particularly preferably 3 to 50% by weight, based on the total weight of component c) and also optionally d) and e) including diluents.

The addition of component c) and also optionally d) and e) takes place preferably at temperatures of −20 to 60° C., preferably 0 to 50 and particularly preferably from 15 to 40° C.

The degree of chain extension, e.g. the molar ratio of NCO-reactive groups of the components c) and also optionally d) and e) used for the chain extension and chain termination to free NCO groups of the prepolymer is generally ≥50 and ≤150%, preferably ≥50 and ≤120%, particularly preferably ≥60 and ≤100% and very particularly preferably ≥70 and ≤95%.

Preferably, the molar ratio of the isocyanate-reactive groups of component c) to the free NCO groups of the prepolymer is ≥50 and ≤120%, particularly preferably ≥60 and ≤100% and very particularly preferably ≥70 and ≤95%.

In a preferred embodiment of the invention, the free NCO groups of the prepolymer are reacted only partially with component c), the molar ratio of the isocyanate-reactive groups of component c) to the free NCO groups of the prepolymer being preferably ≥60 and ≤95%, and the remaining free NCO groups react to completion with the hydroxy groups of the solvent, giving an NCO-free polyurethane urea.

Following the preparation, the polyurethane urea, if solvents or solvent mixtures according to the invention have already been used in the preparation process, can furthermore be diluted with a solvent or solvent mixture and be thereby dissolved, where the solvent consists of one or more monohydroxy-functional alcohols, or a solvent mixture consisting of organic solvents which comprises ≥50% by weight, based on the total mass of the solvent mixture, of at least one monohydroxy-functional alcohol, is used.

If no solvents or solvent mixtures according to the invention have been used during the reaction, then after the preparation of the polyurethane urea, the latter is used in a solvent or solvent mixture where the solvent consists of one or more monohydroxy-functional alcohols, or a solvent mixture consisting of organic solvents which comprises ≥50% by weight, based on the total mass of the solvent mixture, of at least one monohydroxy-functional alcohol, is used.

The dissolution of the polyurethane urea can take place using the customary techniques for shearing, for example by stirring using standard stirrers, for example as specified in DIN 28131.

Suitable solvents or constituents of the solvent mixture are in principle all monohydroxy-functional, aliphatic alcohols with one to six carbon atoms, such as, for example, methanol, ethanol, n-propanol, isopropanol, n-butanol, sec-butanol and/or butyl glycol. The monohydroxy-functional alcohol is particularly preferably ethanol.

If a solvent mixture is used, then in addition to the monohydroxy-functional alcohols, ≤50% by weight, based on the total mass of the solvent mixtures, of a further organic solvent can also be used. Suitable solvents are here are for example esters, such as e.g. ethyl acetate, butyl acetate, methyoxypropyl acetate or butyrolactone, ketones, such as e.g. acetone or methyl ethyl ketone, ethers, such as e.g. tert-butyl methyl ether.

In the event of using ethanol, typical denaturing agents can be present as additives in the customary additive amounts.

Preferably, the fraction of the further organic solvents is ≤30% by weight, particularly preferably ≤5% by weight and very particularly preferably ≤2% by weight, based on the total weight of the solvent mixture. In a very particularly preferred embodiment, no further organic solvents of any kind are present besides monohydroxy-functional, aliphatic alcohols.

The solvent mixture of the composition of the invention contains preferably ≥70% by weight, particularly preferably ≥95% by weight and very particularly preferably ≥98% by weight, based on the total mass of the solvent mixture, of at least one monohydroxy-functional alcohol.

As further solvents unsuitable are physiologically nontolerable solvents such as, for example, dimethylformamide, dimethylacetamide, γ-butyrolactone, N-methyl-2-pyrrolidone or toluene, as are often used as co-solvents for polyurethanes or polyurethane ureas, and these should preferably not be present in compositions applied to skin or plants.

The composition of the invention is therefore preferably free from these solvents.

All solvents used in accordance with the invention are very preferably solvents which are considered physiologically acceptable. Physiologically acceptable solvents are meant, in particular, those which have no current GHS classification (GHS=Global Harmonized System of Classification and Labelling of Chemicals) as GHS05 (irritant), GHS06 (toxic), GHS08 (health hazard), according to the current CLP Regulation (Regulation (EC) No. 1272/2008 on the classification, labelling and packaging of substances and mixtures, in force since 2009). The composition according to the invention is hence preferably free of solvents having a GHS classification as GHS05 (irritant), GHS06 (toxic), GHS08 (health hazard), according to the above mentioned version of the CLP Regulation.

The further solvents are also not water. The polyurethane urea solution obtained by dissolving the polyurethane urea in the solvents or solvent mixtures used according to the invention is preferably anhydrous, with the exception of the fractions of water which are present in the organic solvents used as a consequence of their production.

The water fraction of the polyurethane urea solution is preferably ≤10% by weight, particularly preferably ≤4.5% by weight and very particularly preferably ≤1% by weight, based on the total mass of the polyurethane urea solution.

The fraction of the polyurethane urea (as substance without solvent, also referred to as solids content) in the polyurethane urea solution used according to the invention is here preferably ≥10 and ≤80% by weight, particularly preferably ≥15 and ≤60% by weight and very particularly preferably ≥20 and ≤50% by weight, based on the total weight of the polyurethane urea solution.

The fraction of the polyurethane urea solution used in the composition is preferably ≥0.5 and ≤95% by weight, particularly preferably ≥1 and ≤60% by weight and very particularly preferably ≥2 and ≤40% by weight, based on the total mass of the composition.

The solids content of the polyurethane urea solution is preferably selected such that the compositions contain preferably ≥0.1% by weight and ≤50% by weight, more preferably ≥0.5 and ≤20% by weight and very preferably ≥1 and ≤10% by weight of the polyurethane urea as substance, based on the total mass of the composition.

The composition of the invention further comprises at least one active ingredient and/or fragrance.

The fraction of the active ingredients and/or fragrances in total is preferably ≥0.001 and ≤60% by weight, particularly preferably ≥0.01 and ≤40% by weight and very particularly preferably ≥0.1 and ≤35% by weight, based on the total mass of the composition.

In the context of the invention, active ingredients are defined as elements or chemical compounds which have a specific effect on living systems, more particularly prions, viruses, bacteria, cells, fungi and organisms.

Active ingredients in the sense of the invention encompass active cosmetic ingredients, biocides (for pest control apart from crop protection agents), active pharmacological and veterinary ingredients, active ingredients customary for treating plants, and also active ingredients for the repulsion of animals (repellents). Also possible are active ingredient combinations and their combination with, for example, auxiliaries, binders, neutralizing agents or additives.

In one preferred embodiment of the invention, the active ingredients have ionic groups.

Especially preferred active ingredients are active cosmetic ingredients, very preferably active antiperspirant ingredients.

### Active Cosmetic Ingredients:

Examples of cosmetically active, optionally also therapeutically active, ingredients include the following: anti-acne agents, antimicrobial agents, active antiperspirant ingredients, astringent agents, deodorants, conditioners for the skin, skin-smoothing agents, keratolytics, radical scavengers for free radicals, active antiseptic ingredients, active ingredients to counter skin ageing and/or agents which modulate the differentiation and/or proliferation and/or pigmentation of the skin, vitamins such as vitamin C, active ingredients with a secondary stimulating effect, such as α-hydroxy acids, β-hydroxy acids such as salicylic acid, and also acylated derivatives thereof, 2-hydroxyalkanoic acid and derivatives thereof, α-ketone acids, β-ketone acids, retinoids (retinol, retinal, retinoic acid), anthralins (dioxyanthranol), anthranoids, peroxides (especially benzoyl peroxide), minoxidil, lithium salts, antimetabolites, vitamin D and its derivatives; catechins, flavonoids, ceramides, enzymes, coenzymes such as coenzyme Q10, enzyme inhibitors, skin-calming agents.

The compositions of the invention may further comprise active ingredients based on active plant ingredient extracts, and/or individual substances or extracts obtained from them, such as those selected from the group consisting of solid plant extracts, liquid plant extracts, hydrophilic plant extracts, lipophilic plant extracts, individual plant constituents; and also mixtures thereof, such as flavonoids and their aglyca: rutin, quercetin, diosmin, hyperoside, (neo)hesperidine, hesperitin, ginkgo biloba (e.g. ginkoflavone glycosides), crataegus extract (e.g. oligomeric procyanidins), buckwheat (e.g. rutin), Sophora japonica (e.g. rutin), birch leaves (e.g. quercetin glycosides, hyperoside and rutin), elderflowers (e.g. rutin), linden blossom (e.g. essential oil with quercetin and farnesol), St John's wort oil (e.g. olive oil extract), calendula, arnica (e.g. oily extracts of the flowers with essential oil, polar extracts with flavonoids), Melissa (e.g. flavones, essential oil); immunostimulants: Echinacea purpurea (e.g. alcoholic extracts, fresh plant juice, pressed juice), eleutherococcus senticosus; alkaloids: rauwolfia (e.g. prajmalin), myrtle (e.g. vincamin); further phytopharmaceuticals: aloe, horsechestnut (e.g. aescin), garlic (e.g. garlic oil), pineapple (e.g. bromelains), ginseng (e.g. ginsenosides), milk-thistle fruits (e.g. extract standardized with respect to silymarin), butcher's broom root (e.g. ruscogenin), valerian (e.g. valepotriates, Tct. Valerianae), kava-kava (e.g. kavalactones), hop flowers (e.g. hop bitters), Extr. Passi-florae, gentian (e.g. ethanolic extract), anthraquinone-containing drug extracts, e.g. aloin-containing aloe vera juice, pollen extract, algae extracts, liquorice extracts, palm extract, galphimia (e.g. original tincture), mistletoe (e.g. aqueous-ethanolic extract), phytosterols (e.g. β-sitosterin), verbascum (e.g. aqueous-alcoholic extract), drosera (e.g. vinum liquorosum extract), sea-buckthorn fruits (e.g. juice obtained therefrom or sea-buckthorn oil), marshmallow root, primula root extract, fresh plant extracts of mallow, comfrey, ivory, horsetail, yarrow, ribwort (e.g. pressed juice), stinging nettle, greater celandine, parsley; plant extracts from Norolaena lobata, tagetes lucida, Teeoma siems, momordica charantia, and aloe vera extracts.

Active ingredients which can be used advantageously in the sense of the present invention include water-soluble antioxidants, such as, for example, vitamins, e.g. ascorbic acid and derivatives thereof. Especially advantageous are vitamin E and its derivatives and also vitamin A and its derivatives. All antioxidants that are customary or suitable for cosmetic applications may be used.

Such antioxidants are advantageously selected from the group consisting of amino acids (e.g. glycine, histidine, tyrosine, tryptophan) and derivatives thereof, imidazoles (e.g. urocanic acid) and derivatives thereof, peptides such as D,L-carnosine, D-camosine, L-camosine and derivatives thereof (e.g. anserin), carotenoids, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystin, cystamine and their glycosyl, N-acetyl, methyl, ethyl, propyl, amyl, butyl and lauryl, palmitoyl, oleyl, γ-linoleyl, cholesteryl and glyceryl esters) and also salts thereof, dilauryl thio-dipropionate, distearyl thiodipropionate, thiodipropionoic acid and derivatives thereof (esters, ethers, peptides, lipids, nucleotides, nucleosides and salts) and also sulphoximine compounds (e.g. buthionine sulphoximines, homocysteine sulphoximine, buthionine sulphones, penta-, hexa-, heptathionine sulphoximin) in very low tolerated doses (e.g. pmol to imol/kg), and also (metal)-chelating agents (e.g. α-hydroxy fatty acids, palmitic acid, phytic acid, phytin, lactoferrin), α-hydroxy acids (e.g. citric acid, lactic acid, malic acid, tartaric acid and mandelic acid), humic acid, bile acid, bile extracts, bilirubin, biliverdin, EDTA, EGTA and derivatives thereof, unsaturated fatty acids and derivatives thereof (e.g. γ-ü-nolenic acid, linoleic acid, oleic acid), folic acid and derivatives thereof, ubiquinone and ubiquinol and derivatives thereof, tocopheroles and derivatives (e.g. vitamin E acetate), vitamin A and derivatives (vitamin A palmitate) and also coniferyl benzoate of benzoin resin, rutic acid and derivatives thereof, butylated hydroxytoluene, butylated hydroxyanisole, nordihydroguaiac resin acid, nordihydroguaiaretic acid, trihydroxybutyrophenone, uric acid and derivatives thereof, mannose and derivatives thereof, sesamol, sesamolin, zinc and its derivatives (e.g. ZnO, ZnSO₄), selenium and its derivatives (e.g. selenium methionine), stilbenes and derivatives thereof (e.g. stilbene oxide, trans-stilbene oxide) and the inventively appropriate derivatives (salts, esters, ethers, sugars, nucleotides, nucleosides, peptides and lipids) of these stated active ingredients.

The amount of the aforementioned antioxidants (one or more compounds) in the composition is preferably 0.001 to 30% by weight, particularly preferably 0.05 to 20% by weight, more particularly 0.5 to 10% by weight, based on the total weight of the composition.

If vitamin E and/or derivatives thereof represent the additional antioxidant or antioxidants, it is advantageous to select their respective concentrations from the range from 0.001 to 10% by weight, based on the total weight of the composition.

If vitamin A, and/or vitamin A derivatives, and/or keratins and/or derivatives thereof represent the additional antioxidant or antioxidants, it is advantageous to select their respective concentrations from the range from 0.001 to 10% by weight, based on the total weight of the composition.

The compositions of the invention may further advantageously also comprise self-tanning substances as active ingredients, such as dihydroxyacetone and/or melamine derivatives, for example. They are present preferably in concentrations of 1% by weight to 10% by weight, based on the total weight of the composition.

Active antiperspirant ingredients which can be used are all known compounds with antiperspirant activity that influence the activity of the sebaceous glands. Preferred are the aluminum-based active antiperspirant ingredients, of which, advantageously, acidic aluminium salts can be used in aqueous solution. The concentration ranges described refer in this context to the so-called active contents of the antiperspirant complexes, and to anhydrous complexes in the case of the aluminum compounds. Also preferred, moreover, is the use of what are called activated aluminum chlorohydrates. Active antiperspirant ingredients are, for example, the following:

Aluminum salts of the empirical formula [Al₂(OH)ₘClₙ] (where m+n=6) (aluminum chlorohydrate (ACH)):
aluminum chlorohydrate [Al₂(OH)₅Cl]×H₂O standard aluminum complexes: Locron L, Locron LIC, Locron LIF (Clariant), Chlorhydrol (Reheis), ACH-303 (Summit), Aloxicoll L (Giulini). Activated aluminum complexes: Reach 501 (Reheis), Aloxicoll 51 L
Aluminum sesquichlorohydrate [Al₂(OH)₄0.5Cl_{1.5}]×H₂O standard aluminum complexes: Aloxicoll 31 L (Giulini), Westchlor 186 (Westwood Chemicals). Activated aluminum complexes: Reach 301 (Reheis)
Aluminum dichlorohydrate [Al₂(OH)₄Cl₂]×H₂O

Aluminum zirconium salts as well, however, such as aluminum zirconium-tetrachloroglycine complexes (ZAG), are aluminum-based active antiperspirant ingredients that are suitable in accordance with the invention.

Particularly preferred active antiperspirant ingredients are aluminum chlorohydrate (ACH) and the aluminum zirconium-tetrachloroglycine complex (ZAG), very preferably ACH.

The active antiperspirant ingredients may be used in the compositions of the invention in an amount of ≥0.1% by weight and ≤35% by weight, preferably of ≥1% by weight and ≤30% by weight, more particularly ≥5% by weight and ≤20% by weight, based in each case on the total amount of the composition.

Customary deodorants as well may advantageously be added to the compositions of the invention. All active ingredients commonplace for deodorants may be advantageously utilized, examples being odor concealers such as the usual perfume ingredients, odor absorbers, examples being the phyllosilicates described in DE 40 09 347, and of these in particular montmorillonite, kaolinite, ilite, beidellite, nontronite, saponite, hectorite, bentonite, smectite, and also, for example zinc salts of ricinolic acid.

Antimicrobial agents are likewise suitable for incorporation into the compositions of the invention. Advantageous substances are, for example, 2,4,4′-trichloro-2′-hydroxdiphenyl ether (irgasan), 1,6-di-(4-chlorophenylbiguanido)hexane (chlorhexidine), 3,4,4′-trichlorocarbanilide, polyhexamethylenebiguanide (PHMB), quaternary ammonium compounds, clove oil, mint oil, thyme oil, triethyl citrate, farnesol (3,7,11-trimethyl-2,6,10-dodecatrien-1-ol) and also the active agents described in DE 37 40 186, DE 39 38 140, DE 42 04 321, DE 42 29 707, DE 42 29 737, DE 42 37 081, DE 43 09 372 and DE 43 24 219. The amount of the deodorants in the compositions is preferably ≥0.01 and ≤10% by weight, more preferably ≥0.05 and ≤5% by weight, based on the total weight of the composition.

### Biocides:

Active biocidal ingredients are, in particular, algicides against algae, acaricides against mites or ticks, bactericides against bacteria, fungicides against fungi, insecticides against insects, microbicides treatment against germs, molluscicides against molluscs, nematicides against threadworms (nematodes), rodenticides against rodents, virucides against viruses, antibiotics, or spermicides. Specific examples of fungicides, bactericides, insecticides, acaricides and nematicides are likewise the compounds stated later on below as corresponding active ingredients for crops. Use may also be made, moreover, of active growth-inhibiting ingredients, examples being bacteriostatics or virostatics.

### Active Pharmacological Ingredients:

Active pharmaceutical or therapeutic ingredients are those which in the sense of German pharmaceutical law are intended for purposes including the healing, allevation or prevention of diseases, complaints, physical damage or pathological conditions. The active ingredients are intended in particular for external application, and may be skin-active ingredients or else transdermal active ingredients. They include, for example, the following: agents for the treatment of skin diseases, such as active antibacterial ingredients, antimycotics, such as terbinafine, antiviral ingredients, active anti-inflammatory ingredients, such as dexpanthenol, active anti-itching ingredients, cortisone and derivatives, such as glucocorticoids, such as prednisone, prednisolone, methylprednisolone, betamethasone, dexamethasone, triamcinolone, paramethasone and fludrocortisone, agents for the treatment of skin diseases, such as of neurodermatitis, of atopic dermatitis, etc., and anti-herpes agents such as virostatics.

The composition of the invention may further comprise additional constituents customary for the intended purpose, such as additives which promote the release of active pharmacological ingredients (“drug-eluting additives”).

### Active Veterinary Ingredients:

Active veterinary ingredients in the sense of the invention are, in particular, active ingredients against ectoparasites. Examples of suitable such active ingredients include the following:

from the class of amidine derivatives, for example:
amitraz, chlormebuform, cymiazole, demiditraz;
from the class of arylisoxazolines, not excluding related classes with pyrroline or pyrrolidine moiety replacing the isoxazoline ring, for example:
afoxolaner, fluralaner;
from the class of bacillus thuringiensis strains, for example:
bacillus thuringiensis strains;
from the class of benzoylureas, for example:
bistrifluron, chlofluazuron, chlorfluazuron, diflubenzuron, fluazuron, flucycloxuron, flufenoxuron, hexaflumuron, lufenuron, novaluron, noviflumuron, penfluron, teflubenzuron, triflumuron;
from the class of beta-ketonitrile derivatives, for example:
cyenopyrafen, cyflumetofen;
from the class of carbamates, for example:
alanycarb, aldicarb, aldoxycarb, allyxycarb, aminocarb, bendiocarb, benfuracarb, bufencarb, butacarb, butocarboxim, butoxycarboxim, carbaryl, carbofuran, carbosulfan, cloethocarb, dimetilan, ethiofencarb, fenobucarb, fenothiocarb, formetanate, formparanate, furathiocarb, isoprocarb, metam-sodium, methiocarb, methomyl, metolcarb, oxamyl, pirimicarb, promecarb, propoxur, thiodicarb, thiofanox, triazamate, trimethacarb, xmc, xylylcarb;
from the class of chloronicotinyls, for example:
acetamiprid, clothianidin, dinotefuran, flupyradifurone, imidacloprid, nicotine, nitenpyram, nithiazine, thiacloprid, thiamethoxam;
from the class of diacylhydrazines, for example:
chromafenozide, halofenozide, methoxyfenozide, tebufenozide;
from the class of diamides, for example:
chlorantraniliprole, cyantraniliprole;
from the class of dicarboxamides, for example:
flubendiamide;
from the class of dinitrophenols, for example:
binapacyrl, dinobuton, dinocap, dnoc;
from the class of feeding inhibitors, for example:
cryolite, flonicamid, pymetrozine;
from the class of fumigants, for example:
aluminium phosphide, methyl bromide, sulphuryl fluoride;
from the class of halogenated carbonhydrogen compounds (hch), for example:
ddt, methoxychlor;
from the class of macrocyclic lactones, for example:
moxidectin, emamectin benzoate, latidectin, lepimectin;
from the class of microorganisms, for example:
bacillus spec., beauveria spec., metarrhizium spec., paecilomyces spec., verticillium spec.;
from the class of mite growth inhibitors, for example:
amidoflumet, benclothiaz, benzoximate, bifenazate, bromopropylate, chlordimeform, chlorobenzilate, chloropicrin, clofentezine, clothiazoben, cycloprene, dicyclanil, etoxazole, fenoxacrim, fentrifanil, flubenzimine, flufenerim, flutenzin, gossyplure, hexythiazox, hydramethylnone, japonilure, metoxadiazone, petroleum, potassium oleate, pyridalyl, quinomethionate, tetrasul, triarathene;
from the class of natural products, for example:
codlemone, essential oils, thuringiensin;
from the class of neem components, for example:
azadirachtin a;
from the class of nereistoxin analogues, for example:
bensultap, cartap, sulfoxaflor, thiocyclam, thiocyclam hydrogen oxalate, thiosultap sodium, thiosultap-sodium;
from the class of organic acids, for example:
formic acid, oxalic acid;
from the class of organochlorines, for example:
camphechlor, chlordane, endosulfan, γ-hch, hch, heptachlor, lindane;
from the class of organophosphates, for example:
acephate, aromfenvinfos (-methyl), aromophos-ethyl, autathiofos, azamethiphos, azinphos (-methyl, -ethyl), cadusafos, carbophenothion, chlorethoxyfos, chlorfenvinphos, chlormephos, chlorpyrifos (-methyl/-ethyl), cyanofenphos, cyanophos, demeton-s-methyl, demeton-s-methylsulphone, dialifos, diazinon, dichlofenthion, dichlorvos/ddvp, dicrotophos, dimethoate, dimethylvinphos, dioxabenzofos, disulfoton, epn, ethion, ethoprophos, etrimfos, famphur, fenamiphos, fenitrothion, fensulfothion, fenthion, flupyrazofos, fonofos, formothion, fosmethilan, fosthiazate, heptenophos, iodofenphos, iprobenfos, isazofos, isofenphos, isopropyl o-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidone, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulfotep, sulprofos, tebupirimfos, temephos, terbufos, tetrachlorvinphos, thiometon, triazophos, triclorfon, vamidothion;
from the class of organotin compounds, for example:
azocyclotin, cyhexatin, fenbutatin-oxide;
from the class of other decouplers, for example:
sulfluramid;
from the class of other inhibitors of cuticle development, for example:
buprofezin, cyromazine; from the class of other inhibitors of cuticle development, for example:
buprofezin, cyromazine;
from the class of others, for example:
chinomethionat, pyrifluquinazon;
from the class of oxadiazines, for example:
indoxacarb;
from the class of phenylpyrazoles, for example:
acetoprole, ethiprole, fipronil, pyrafluprole, pyriprole, vaniliprole;
from the class of pyrethroids, for example:
acrinathrin, allethrin (d-cis-trans, d-trans-), β-cyfluthrin, bifenthrin, bioallethrin, bioallethrin-s-cyclopentyl-isomer, bioethanomethrin, biopermethrin, bioresmethrin, chlovaporthrin, cis-cypermethrin, cis-permethrin, cis-resmethrin, clocythrin, cycloprothrin, cyfluthrin, cyhalothrin (λ-), cypermethrin (α-, β-, θ-, ζ-), cyphenothrin, deltamethrin, empenthrin (Ir-isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, γ-cyhalothrin, imiprothrin, kadethrin, -cyhalothrin, metofluthrin, permethrin (cis-, trans-), phenothrin (Ir-trans isomer), prallethrin, profluthrin, protrifenbute, pyresmethrin, pyrethrins (pyrethrum), resmethrin, ru 15525, silafluofen, tau-fluvalinate, tefluthrin, terallethrin, tetramethrin (-1r-isomer), tralomethrin, transfluthrin, zxi 8901;
from the class of pyrroles, for example:
chlorfenapyr;
from the class of quinones, for example:
acequinocyl;
from the class of rotenone, for example:
rotenone;
from the class of semicarbazones, for example:
metaflumizone;
from the class of spinosynes, for example:
spinetoram, spinosad;
from the class of tetronic and tetramic acids, for example:
spirodiclofen, spiromesifen, spirotetramat;
from the class of nereistoxin analogues, for example:
bensultap, cartap, sulfoxaflor, thiocyclam, thiocyclam hydrogen oxalate, thiosultap sodium, thiosultap-sodium;
from diverse other classes, for example:
amoscanate, bephenium, bunamidine, clonazepam, clorsulon, diamfenetide, dichlorophen, diethylcarbamazine, emetine, hetolin, hycanthone, lucanthone, miracil, mirasan, niclosamide, niridazole, nitroxynile, nitroscanate, oltipraz, omphalotin, oxamniquine, paromomycin, piperazine, resorantel.

Salts like hydrochlorides, tartrates, citrates, embonates/pamoates or benzoates are included.

### Substances for Treating Plants:

The active ingredients which can be used in the compositions of the invention may be for example, but not conclusively, all substances customary for treatment of plants; with preference, mention may be made of fungicides, bactericides, insecticides, akaricides, nematicides, herbicides, plant growth regulators or plant nutrients.

In the sense of the invention, plants are taken to include the corresponding seed as well.

Examples of fungicides include the following:

2-Anilino-4-methyl-6-cyclopropyl-pyrimidine; 2′,6′-dibromo-2-methyl-4′-trifluoromethoxy-4-trifluoromethyl-1,3-thiazole-5-carboxanilide; 2,6-dichloro-N-(4-trifluoromethylbenzyl)-benzamide; (E)-2-methoximino-N-methyl-2-(2-phenoxyphenyl)-acetamide; 8-hydroxyquinoline sulphate; methyl (E)-2-{2-[6-(2-cyanophenoxy)-pyrimidin-4-yloxy]-phenyl}-3-methoxyacrylate; methyl-(E) methoximino [α-(o-tolyloxy)-o-tolyl]-acetate; 2-phenylphenol (OPP), aldimorph, ampropylfos, anilazine, azaconazole, benalaxyl, benodanil, benomyl, binapacryl, biphenyl, bitertanol, blasticidin-S, bromuconazole, bupirimate, buthiobate, calcium polysulfide, captafol, captan, carbendazim, carboxin, chinomethionate (quinomethionate), chloroneb, chloropierin, chlorothalonil, chlozolinate, cufraneb, cymoxanil, cyproconazole, cyprofuram, carpropamide, dichlorophene, diclobutrazole, dichlofluanide, diclomezine, diclorane, diethofencarb, difenoconazole, dimethirimol, dimethomorph, diniconazole, dinocap, diphenylamine, dipyrithione, ditalimfos, dithianone, dodine, drazoxolon, edifenphos, epoxyconazole, ethirimol, etridiazole, fenarimol, fenbuconazole, fenfuram, fenitropan, fenpiclonil, fenpropidin, fenpropimorph, fentin acetate, fentin hydroxide, ferbam, ferimzone, fluazinam, fludioxonil, fluoromide, fluquinconazole, flusilazole, flusulfamide, flutolanil, flutriafol, folpet, fosetyl-aluminum, fthalide, fuberidazole, furalaxyl, furmecyclox, fenhexamid, guazatine, hexachlorobenzene, hexaconazole, hymexazole, imiazalil, imibenconazole, iminoctadine, iprobenfos (IBP), iprodione, isoprothiolane, iprovalicarb, kasugamycin, copper preparations, such as: copper hydroxide, copper naphthenate, copper oxychloride, copper sulfate, copper oxide, oxine-copper and Bordeaux mixture, mancopper, mancozeb, maneb, mepanipyrim, mepronil, metalaxyl, metconazole, methasulfocarb, methfuroxam, metiram, metsulfovax, myclobutanil, nickel dimethyldithiocarbamate, nitrothal-isopropyl, nuarimol, ofurace, oxadixyl, oxamocarb, oxycarboxin, pefurazoate, penconazole, pencycuron, phosdiphen, pimaricin, piperalin, polyoxin, probenazole, prochloraz, procymidon, propamocarb, propiconazole, propineb, pyrazophos, pyrifenox, pyrimethanil, pyroquilon, quintozene (PCNB), quinoxyfen, sulphur and sulphur preparations, spiroxamine, tebuconazole, tecloftalam, tecnazene, tetraconazole, thiabendazole, thicyofen, thiophanate-methyl, thiram, tolclophos-methyl, tolylfluanid, triadimefon, triadimenol, triazoxide, trichlamide, tricyclazole, tridemorph, triflumizole, triforine, triticonazole, trifloxystrobin, validamycin A, vinclozolin, zineb, ziram, and 2-[2-(1-chloro-cyclopropyl)-3-(2-chlorophenyl)-2-hydroxypropyl]-2,4-dihydro-[1.2.4]-triazole-3-thione.

### Examples of Bactericides Include:

Bronopol, dichlorophen, nitrapyrin, nickel-dimethyldithiocarbamate, kasugamycin, octhilinone, furancarboxylic acid, oxytetracycline, probenazole, streptomycin, tecloftalam, copper sulfate and other copper preparations.

Examples of insecticides, acaricides and nematicides include:

Abamectin, acephate, acetamiprid, acrinathrin, alanycarb, aldicarb, alphamethrin, amitraz, avermectin, AZ 60541, azadirachtin, azinphos A, azinphos M, azocyclotin, Bacillus thuringiensis, 4-bromo-2-(4-chlorophenyl)-1-(ethoxymethyl)-5-(trifluoromethyl)-1H-pyrrole-3-carbonitrile, bendiocarb, benfuracarb, bensultap, betacyfluthrin, bifenthrin, BPMC, brofenprox, bromophos A, bufencarb, buprofezin, butocarboxin, butylpyridaben, cadusafos, carbaryl, carbofuran, carbophenothion, carbosulfan, cartap, chloethocarb, chlorethoxyfos, chlorfenvinphos, chlorfluazuron, chlormephos, N-[(6-chloro-3-pyridinyl)-methyl]-N′-cyano-N-methyl-ethanimidamide, chlorpyrifos, chlorpyrifos M, cis-resmethrin, clocythrin, clofentezine, clothianidin, cyanophos, cycloprothrin, cyfluthrin, cyhalothrin, cyhexatin, cypermethrin, cyromazine, deltamethrin, demeton-M, demeton-S, demeton-S-methyl, diafenthiuron, diazinon, dichlofenthion, dichlorvos, dicliphos, dicrotophos, diethion, diflubenzuron, dimethoate, dimethylvinphos, dioxathion, disulfoton, emamectin, esfenvalerate, ethiofencarb, ethion, ethofenprox, ethoprophos, etrimphos, fenamiphos, fenazaquin, fenbutatin oxide, fenitrothion, phenobucarb, phenothiocarb, phenoxycarb, fenpropathrin, fenpyrad, fenpyroximate, fenthion, fenvalerate, fipronil, fluazuron, flucycloxuron, flucythrinate, flufenoxuron, flufenprox, fluvalinate, fonophos, formothion, fosthiazate, fubfenprox, furathiocarb, HCH, heptenophos, hexaflumuron, hexythiazox, imidacloprid, iprobenfos, isazophos, isofenphos, isoprocarb, isoxathion, ivermectin, -cyhalothrin, lufenuron, malathion, mecarbam, mevinphos, mesulfenphos, metaldehyde, methacrifos, methamidophos, methidathion, methiocarb, methomyl, metolcarb, milbemectin, monocrotophos, moxidectin, naled, NC 184, nitenpyram, omethoate, oxamyl, oxydemethon M, oxydeprofos, parathion A, parathion M, permethrin, phenthoate, phorate, phosalone, phosmet, phosphamidon, phoxim, pirimicarb, pirimiphos M, pirimiphos A, profenophos, promecarb, propaphos, propoxur, prothiophos, prothoate, pymetrozine, pyrachlophos, pyridaphenthion, pyresmethrin, pyrethrum, pyridaben, pyrimidifen, pyriproxifen, quinalphos, salithion, sebufos, silafluofen, sulfotep, sulprofos, tebufenozide, tebufenpyrad, tebupirimiphos, teflubenzuron, tefluthrin, temephos, terbam, terbufos, tetrachlorvinphos, thiacloprid, thiafenox, thiamethoxam, thiodicarb, thiofanox, thiomethon, thionazin, thuringiensin, tralomethrin, transfluthrin, triarathene, triazophos, triazuron, trichlorfon, triflumuron, trimethacarb, vamidothion, XMC, xylylcarb, zetamethrin.

Especially preferably, the powder formulations of the invention comprise imidacloprid, thiacloprid, thiamethoxam, acetamiprid, clothianidin, betacyfluthrin, cypermethrin, transfluthrin, lambda-cyhalothrin and/or azinphosmethyl.

Examples of herbicides include:

Anilides, such as diflufenican and propanil; arylcarboxylic acids, such as dichloropicolinic acid, dicamba and picloram; aryloxyalkanoic acids, such as 2,4-D, 2,4-DB, 2,4-DP, fluroxypyr, MCPA, MCPP and triclopyr; aryloxy-phenoxy-alkanoic acid esters, such as diclofop-methyl, fenoxaprop-ethyl, fluazifop-butyl, haloxyfop-methyl and quizalofop-ethyl; azinones, such as chloridazon and norflurazon; carbamates, such as chlorpropham, desmedipham, phenmedipham and propham; chloroacetanilides, such as alachlor, acetochlor, butachlor, metazachlor, metolachlor, pretilachlor and propachlor; dinitroanilines, such as oryzalin, pendimethalin and trifluralin; diphenyl ethers, such as acifluorfen, bifenox, fluoroglycofen, fomesafen, halosafen, lactofen and oxyfluorfen; ureas, such as chlortoluron, diuron, fluometuron, isoproturon, linuron and methabenzthiazuron; hydroxylamines, such as alloxydim, clethodim, cycloxydim, sethoxydim and tralkoxydim; imidazolinones such as imazethapyr, imazamethabenz, imazapyr and imazaquin; nitriles, such as bromoxynil, dichlobenil and ioxynil; oxyacetamides, such as mefenacet; sulphonyl ureas, such as amidosulphuron, bensulphuron-methyl, chlorimuronethyl, chlorsulphuron, cinosulphuron, metsulphuron-methyl, nicosulphuron, primisulphuron, pyrazosulphuron-ethyl, thifensulphuron-methyl, triasulphuron and tribenuron-methyl; thiolcarbamates, such as butylate, cycloate, diallate, EPTC, esprocarb, molinate, prosulfocarb, thiobencarb and triallate; triazines, such as atrazine, cyanazine, simazine, simetryne, terbutryne and terbutylazine; triazinones, such as hexazinone, metamitron and metribuzin; miscellaneous compounds, for example aminotriazole, benfuresate, bentazone, cinmethylin, clomazone, clopyralid, difenzoquat, dithiopyr, ethofumesate, fluorochloridone, gluphosinate, glyphosate, isoxaben, pyridate, quinchlorac, quinmerac, sulphosate and tridiphane. Others that may be mentioned are 4-amino-N-(1,1-dimethylethyl)-4,5-dihydro-3-(1-methylethyl)-5-oxo-1H-1,2,4-triazole-1-carboxamide and benzoic acid, 2-((((4,5-dihydro-4-methyl-5-oxo-3-propoxy-1H-1,2,4-triazol-1-yl)carbonyl)amino)sulfonyl)-methyl ester.

Examples of plant growth regulators include chlorocholine chloride and ethephon.

Examples of plant nutrients include usual inorganic or organic fertilizers for supplying plants with macro- and/or micro-nutrients.

### Repellent:

A repellent is an active ingredient for repelling animals, which is perceived by an organism via its sense of smell and which frightens it off without killing.

Repellents are used in particular against blood-sucking, disease-transmitting arthipods such as mosquitoes, mites, horseflies or ticks.

Examples of natural repellents include essential oils of various representatives of the Lamiaceae (e.g. basal, mint, lavender, sage or thyme), Myrtaceae (e.g. eucalyptuses, clove and tree tea), aromatic true grasses (e.g. citronella, lemon grass and palmarosa) and also the pelargoniums and cedars. Another plant-based active ingredient is, for example, PMD (p-menthane-3,8-diol, also Citriodiol).

Examples of artificial repellents are N,N-diethyl-m-toluamide (DEET), 2-butyl (2-hydroxyethyl)piperidine-1-carboxylate (available under the trade name Saltidin), dimethyl phthalate (trade name: Palatinol M, DMP) or Repellent 3535 (ethyl 3-(N-n-butyl-N-acetylamino)propionate).

Fragrances in the sense of the invention encompass all natural and synthetic substances which develop an odur which is perceived olfactorily.

It is advantageous in accordance with the invention if the composition of the invention comprises one or more fragrances selected from the group of the compounds Limonen [5989-27-5], Citral, Linalool [78-70-6], alpha-isomethylionone [1335-46-2], Geraniol [106-24-1], Citronellol [106-22-9], [24851-98-7], [18479-58-8], [54464-57-2], [80-54-6], [1222-05,5], [32388-55-9], [105-95-3], [31906-04-4], [8008-57-9], [32210-23-4], [120-57-0], [115-95-7], [101-86-0], [140-11-4], [6259-76-3] and [127-51-5], 2-isobutyl-4-hydroxy-4-methyltetrahydropyran, 2-tert-pentylcyclohexyl acetate, 3-methyl-5-phenyl-1-pentanol, 7-acetyl-1,1,3,4,4,6-hexamethyltetralin, diethyl adipate, alpha-amylcinnamaldehyde, alpha-methylionone, amyl C, butylphenylmethylpropional cinnamal, amyl salicylate, amylcinnamyl alcohol, anisyl alcohol, benzoin, benzyl alcohol, benzyl benzoate, benzyl cinnamate, benzyl salicylate, bergamot oil, bitter orange oil, butylphenylmethylpropioal, cardamom oil, cedrol, cinnamyl, cinnamyl alcohol, citronellylmethyl crotonate, lemon oil, coumarin, diethyl succinate, d-limonenes, ethyllinalool, eugenol, Evernia furfuracea extract, Evernia prunastri extract, farnesol, guaiac wood oil, hexylcinnamal, hexyl salicylate, hydroxycitronellal, hydroxyisohexyl S-cyclohexenecarboxaldehyde, lavender oil, lemon oil, linayl acetate, mandarin oil, menthyl PCA, methylheptenone, muscat kernel oil, rosemary oil, sweet orange oil, terpineol, tonkabohnen oil, triethyl citrate and/or vanillin.

It is advantageous in accordance with the invention if the composition of the invention comprises one or more fragrances in a total concentration of ≥0.001 and ≤30% by weight, preferably of ≥0.01 and ≤5% by weight and further preferably ≥0.05 and <1% by weight, based on the total weight of the composition.

In one embodiment of the invention, the compositions of the invention can take no phyllosilicates.

The compositions preferably include a water fraction of ≥0 and ≤30% by weight, particularly preferably of ≥0 and ≤20% by weight, very particularly preferably of ≥0 and ≤5% by weight and further preferably ≥0 and <2% by weight. With more particular preference the cosmetic compositions are water-free, therefore containing no more water than that introduced unavoidably into the formulation via the raw materials as part of the preparation process.

The compositions further preferably comprise customary additives such as emulsifiers, interface-active substances, defoamers, thickeners, surfactants, humectants, filler, film formers, solvents, coalescents, gel formers and/or other polymer dispersions such as, for example, dispersions based on polyacrylates, fillers, plasticizers, pigments, dyes, flow control agents, thixotropic agents, sleekness agents, preservatives, sensory additives, oils, waxes and/or propellants such as, for example, propane/butane or dimethyl ether, etc. The amounts of the various additives are known to the skilled person for the field to be employed, and are situated, for example, in the range of ≥0 and ≤40% by weight, preferably ≥0.1 and ≤40% by weight, based on the total weight of the composition.

In one embodiment of the invention, the composition of the invention comprises humectants as additives.

Advantageous humectants (moisturizers) in the sense of the present invention are, for example, lactic acid and/or lactates, more particularly sodium lactate, butylene glycol, propylene glycol, biosaccharide gum-1, Glycine soya, ethylhexyloxyglycerol, pyrrolidone carboxylic acid and urea. Particularly advantageous, furthermore, is the use of polymeric moisturizers from the group of polysaccharides which are water-soluble and/or swellable in water and/or gelable with the aid of water. Especially advantageous, for example, are hyaluronic acid, chitosan and/or a fucose-rich polysaccharide which is recorded in Chemical Abstracts under the registry number 178463-23-5 and is available for example under the name Fucogel®1000 from the company SOLABIA S.A. Moisturizers may advantageously also be used as anti-wrinkle ingredients for protection from changes in the skin, of the kind occurring in skin ageing, for example.

It is advantageous in the sense of the present invention if the preparation of the invention comprises one or more humectants in a total concentration of 0.1 to 20% by weight and preferably in a total concentration of 0.5 to 10% by weight, based in each case on the total weight of the preparation.

The compositions of the invention may have a variety of consistencies; semi-solid, more particularly in the form of gels, or of low viscosity, more particularly as sprayable compositions, aerosols or oils.

The compositions of the invention preferably immobilize the fragrances or active ingredients on substrates or on the body, or bring about controlled and/or delayed release of the fragrances or active ingredients.

The composition of the invention may preferably be a crop protection composition, a perfume, a deodorant, a repellent product, a skin cosmetology article, a hair cosmetology article, a medical equipment article, an article for clothing, an article for vehicle construction, an article for sports equipment, an article for the construction industry, an article for the electronics industry, an article for cleaning, an article for producing consumer goods, a form of packaging, a container, or else is used in such applications. Particularly preferred are a perfume, a deodorant or a repellent product, very preferably a perfume or a deodorant.

Typical product forms for repellent products for application on the skin are solution, aerosol spray, pump spray, foam, stick, roller, emulsion, gel, and impregnated wipes.

Repellent products customarily comprise film formers, active repellent ingredients and optionally further cosmetic and/or dermatological active ingredients, auxiliaries and adjuvants.

With particular preference the composition of the invention is a deodorant or a perfume, very preferably a deodorant, comprising an active antiperspirant ingredient, or else is used in a deodorant or a perfume, very preferably in a deodorant, comprising an active antiperspirant ingredient.

Deodorants may take the form of aerosols, and also products sprayable by means of a pump device or from squeeze bottles, or else may take the form of rollers, sticks, creams, powders, crystals or impregnated wipes.

Deodorants may be employed, for example, by applications of the armpits, the feet and/or the hands.

Depending on the form of application, deodorants may include not only the aforementioned active ingredients but also fat-restoring and fixing components customary for deodorants, and also fragrances, care compounds and propellants (propane, butane and isobutane), glycols, solubilizers, gel formers, emulsifiers and/or soaps.

The active ingredients in deodorants are often in the form of alcoholic or aqueous-alcoholic solutions.

Perfumes in the sense of the invention include not only products for spray application to the body but also room perfumes, which can be distributed in a room by spraying, for example.

Besides fragrances, perfumes may comprise, in particular, alcohols and/or distilled water.

A further subject of the invention is the composition of the invention for treating the human or animal body.

The composition of the invention may be used in products for treating fungically diseased feet or nails, or in veterinary products which control ectoparasites.

Likewise, a subject of the invention is the use of the composition of the invention to coat substrates, the substrates not being the human or animal body. The materials used in accordance with the invention may be sheet-like textile structures such as, for example, woven fabrics, knitted fabrics, bonded and unbonded nonwovens, or leather, wood, metals, plastics and paper, and also the surfaces of plants. With particular preference the substrates are the surfaces of plants.

Additionally, the use of the composition of the invention for application to the human or animal body, the active ingredient not having a therapeutic effect, is a subject of the invention.

Especially preferred examples of such uses are the perfumes or deodorants set out above.

The compositions of the invention can be used advantageously in all applications where the controlled and possibly also delayed delivery of the active ingredients and fragrances is required, in what are called controlled-release applications.

The invention further encompasses a method for the coating of substrates which is characterized in that a composition of the invention is applied to a substrate, and also encompasses the substrates obtainable by this method.

With regard to the definition of substrates, the embodiments stated above are applicable.

The invention likewise encompasses a method for coating the human and animal body, said method being characterized in that a composition of the invention is applied to the body. In this context, the active ingredients used in the composition of the invention preferably have no therapeutic effect.

The composition here may be applied to parts of or the entire surface of the substrates or body, by means of spraying, dipping, knife coating, spreading with brushes, or printing, for example.

The composition advantageously remains on the substrate or body. Likewise advantageously, the composition forms a film on the substrate or the body. Film forming in this case is accomplished preferably by evaporation of the solvent or solvent mixture.

The invention likewise encompasses a method for reducing perspiration, in which the composition of the invention is applied to the surface of the skin. This is a cosmetic method. Application takes place preferably to the armpits, to the feet or hands, preferably to the armpits.

The present invention is elucidated using the following examples.

### EXAMPLES

Unless noted otherwise, all percentages are by weight.

Unless noted otherwise, all analytical measurements relate to temperatures at 23° C.

The solids contents (non-volatile fraction) were determined according to DIN-EN ISO 3251.

NCO contents were determined, unless expressly stated otherwise, volumetrically in accordance with DIN-EN ISO 11909.

Checking for free NCO groups was carried out by means of IR spectroscopy (band at 2260 cm⁻¹).

The reported viscosities were determined by means of rotary viscometry in accordance with DIN 53019 at 23° C. using a rotary viscometer from Anton Paar Germany GmbH, Ostfildern, DE.

The number-average molecular weight was determined by gel permeation chromatography (GPC) in tetrahydrofuran at 23° C. The procedure used is in line with DIN 55672-1: “Gel permeation chromatography, Part 1—Tetrahydrofuran as eluent” (SECurity GPC System from PSS Polymer Service, flow rate 1.0 ml/min; columns: 2×PSS SDV linear M, 8×300 mm, 5 μm; RID detector). Polystyrene samples of known molar mass are used here for calibration. The calculation of the number-average molecular weight takes place with software support. Base line points and evaluation limits are specified in accordance with DIN 55672 Part 1.

The turbidity values [NTU] were determined by scattered light measurement at a 90° angle (Nephelometry) with a measuring radiation wavelength of 860 nm in accordance with DIN EN ISO 7027, carried out at 23° C. using a laboratory turbidity instrument model 2100AN from HACH LANGE GmbH, Berlin, Germany.

### Substances Used and Abbreviations:

PolyTHF® 2000: Polytetramethylene glycol polyol, OH number 56 mg KOH/g, number-average molecular weight 2000 g/mol (BASF AG, Ludwigshafen, DE)
PolyTHF® 1000: Polytetramethylene glycol polyol, OH number 112 mg KOH/g, number-average molecular weight 1000 g/mol (BASF AG, Ludwigshafen, DE)
Ethanol Unless otherwise noted, ethanol denatured with MEK was used, from Nordbrand, Norhausen, DE.

Other polymeric polyols and isocyanates were used from Covestro AG, Leverkusen, DE. Other chemicals from Sigma-Aldrich Chemie GmbH, Taufkirchen, DE. Unless otherwise mentioned, the raw materials were used without further purification or pretreatment.

### Example 1: Preparation of a Polyurethane Solution in Ethanol (Inventive)

226.2 g of polypropylene glycol having a number-average molecular weight of 2000 g/mol, 62.5 g of polypropylene glycol having a number-average molecular weight of 1000 g/mol were dewatered under a membrane pump vacuum at 100° C. for an hour in a standard stirring apparatus, and then the mixture was introduced under nitrogen at 80° C. Subsequently, at 80° C., 83.4 g of isophorone diisocyanate were added over 5 minutes, and the mixture was stirred at 120° C. for 6 hours until the NCO value was below the theoretical value (which is the point, of conversion of all groups which are reactive towards isocyanate). The prepolymer was cooled to 40° C. and dissolved in 280 g of ethanol and then the temperature was reduced to 18° C. At this point a solution of 34.1 g of methylenebis(4-aminocyclohexane) and 120 g of ethanol was metered in over the course of 30 minutes. A further 4.5 g of methylenebis(4-aminocyclohexane) were added, and then stirring was continued until free isocyanate groups were no longer detectable by IR spectroscopy.

The clear, storage-stable solution obtained had the following properties:

Solids content: 49.8%
Viscosity (viscometer, 23° C.): 1100 mPas

### Example 2: Preparation of a Polyurethane Solution in Ethanol (Inventive)

300 g of POLYTHF 1000 were dewatered under a membrane pump vacuum at 100° C. for an hour in a standard stirring apparatus and then introduced under nitrogen at 80° C. Subsequently, at 80° C., 133.44 g of isophorone diisocyanate were added over 5 minutes, and the mixture was stirred at 110° C. until (about 3 hours) the NCO value was below the theoretical value. The prepolymer was cooled to 40° C. and dissolved in 517 g of ethanol (denatured with MEK) and then the temperature was reduced to 16° C. At this point a solution of 58.8 g of methylenebis(4-aminocyclohexane) and 222 g of ethanol (denatured with MEK) was metered in over the course of 30 minutes, after which a further 410 g of ethanol were added. Stirring continued, until free isocyanate groups were no longer detectable by IR spectroscopy.

The clear, storage-stable solution obtained had the following properties:

Solids content: 30.2%
Viscosity (viscometer, 23° C.): 85 000 mPas

### Example 3: Preparation of a Polyurethane Solution in Ethanol (Inventive)

211 g of POLYTHF 2000 and 52.7 g of POLYTHF 1000 were dewatered under a membrane pump vacuum at 100° C. for an hour in a standard stirring apparatus, then 5.4 g of neopentyl glycol were added, and the mixture was introduced under nitrogen at 80° C. Subsequently, at 80° C., 93.4 g of isophorone diisocyanate were added over 5 minutes, and the mixture was stirred at 110° C. until (about 3 hours) the NCO value was below the theoretical value. The prepolymer was cooled to 40° C. and dissolved in 420 g of ethanol (denatured with diethyl phthlate) and then the temperature was reduced to 17° C. At this point a solution of 35.3 g of methylenebis(4-aminocyclohexane) and 180 g of ethanol (denatured with diethyl phthlate) was metered in over the course of 30 minutes. A further 0.67 g of methylenebis(4-aminocyclohexane) was added, and then stirring was continued until free isocyanate groups were no longer detectable by IR spectroscopy. The clear, storage-stable solution obtained had the following properties:

### Solids Content: 40.5%

Viscosity (Viscometer, 23° C.): 7060 mPas

### Example 4: Preparation of a Polyurethane Solution in Ethanol (Inventive)

150 g of POLYTHF 2000 and 37.50 g of POLYTHF 1000 were dewatered under a membrane pump vacuum at 100° C. for an hour in a standard stirring apparatus and then introduced under nitrogen at 80° C. Subsequently, at 80° C., 75.06 g of isophorone diisocyanate were added over 5 minutes, and the mixture was stirred at 110° C. until (about 3 hours) the NCO value was below the theoretical value. The prepolymer was cooled to 40° C. and dissolved in 630.4 g of ethanol (denatured with diethyl phthalate) and then the temperature was reduced to 15° C. At this point a solution of 37.6 g of methylenebis(4-aminocyclohexane) and 270 g of ethanol (denatured with diethyl phthalate) was metered in over the course of 30 minutes; after a further 30 minutes at 20° C., isocyanate groups were still detectable by IR spectroscopy.

Stirring continued at 23° C. for about 16 hours, until free isocyanate groups were no longer detectable by IR spectroscopy.

The clear, storage-stable solution obtained had the following properties:

Solids content: 23%
Viscosity (viscometer, 23° C.): 280 mPas

### Turbidity: 1.2 NTU

### Example 5: Preparation of a Polyurethane Solution in Ethanol (Inventive)

160 g of POLYTHF 2000 and 40.0 g POLYTHF 1000, were dewatered under a membrane pump vacuum at 100° C. for an hour in a standard stirring apparatus, and then the mixture was introduced under nitrogen at 80° C. Subsequently, at 80° C., 62.9 g of bis(4,4′0-isocyanatocyclo-hexyl)methane were added over 5 minutes, and the mixture was stirred at 110° C. until (about 3 hours) the NCO value was below the theoretical value. The prepolymer was cooled to 40° C. and dissolved in 595 g of ethanol (denatured with diethyl phthalate) and then the temperature was reduced to 19° C. At this point a solution of 20.2 g of methylenebis(4-aminocyclohexane) and 255 g of ethanol (denatured with diethyl phthalate) was metered in over the course of 30 minutes. A further 4.5 g of methylenebis(4-aminocyclohexane) were added, and then stirring was continued until free isocyanate groups were no longer detectable by IR spectroscopy.

The clear, storage-stable solution obtained had the following properties:

Solids content: 25.2%
Viscosity (viscometer, 23° C.): 3400 mPas

### Example 6: Preparation of a Polyurethane Solution in Ethanol (Comparative)

200 g of a linear, difunctional polycarbonate diol based on 1,6-hexanediol and dimethyl carbonate, with a number-average molecular weight of 2000 g/mol, and 50 g of a linear, difunctional polycarbonate diol based on 1,6-hexanediol, with a number-average molecular weight of 1000 g/mol, were dewatered under a membrane pump vacuum at 100° C. for an hour in a standard stirring apparatus, and then introduced at 80° C. under nitrogen. Subsequently, at 80° C., 66.7 g of isophorone diisocyanate were added over the course of 5 minutes, and the mixture was stirred at 110° C. until (about 3 hours) the NCO value was below the theoretical value. The prepolymer was cooled to 40° C. and dissolved in 720 g of ethanol (denatured with diethyl phthalate), at which point the product did not fully dissolve, and the temperature was subsequently reduced to 17° C. Then a solution of 25.2 g of methylenebis(4-aminocyclohexane) and 310 g of ethanol (denatured with diethyl phthalate) was metered in over the course of 30 minutes, producing a two-phase mixture. Stirring was then continued, with formation not of a stable solution but instead of a two-phase mixture, from which the solid phase underwent sedimentation.

### Comparative Performance Experiments

### Example 7: Perfume Composition

One spray jet of a solution of a mixture of fragrances (Fresh Protect, 0.5% by weight in ethanol) or a mixture of the Fresh Protect solution (0.5% by weight in ethanol) with the polyurethane urea solution according to the invention (amount selected so that the mixture contained 4% by weight of polyurethane urea as substance, based on the overall mixture) was applied to the underarm of individuals from a trial group. The trial individuals then evaluated the intensity of the fragrance directly after spray application, after 1 hour, after 2 hours, after 4 hours and after 6 hours. Evaluation took place in a scale from 0 to 10, with 0 denoting no odor and 10 extremely strong odor. From the evaluations, the average was formed.

The intensity of fragrance odor directly after the spray application was evaluated as 6.75 without the polyurethane urea solution, and 4.50 with polyurethane urea solution. The polyurethane urea solution therefore significantly influences the fragrance intensity.

The FIGURE shows the relative loss in fragrance intensity between directly after spray application and time t. It has clearly been shown that the addition of the polyurethane urea solution from experiment 1 delays the abatement of the fragrance intensity.

### Example 8: Antiperspirant Composition

Compatibility of Active Antiperspirant Ingredients with Film-Formers:

68% by weight of BAYCUSAN C1008 (aqueous polyurethane urea dispersion having a solids content of 30% by weight, available from Covestro AG) and 18% by weight of aqueous aluminum chlorohydrate solution (corresponding to 4% by weight of aluminum chlorohydrate (ACH)+14% by weight of water) were mixed at room temperature with 14% by weight of ethanol. BAYCUSAN C1008 showed no compatibility with ethanol, and flocculation occurred. No film was obtained when the mixture was sprayed onto the skin at room temperature.

68% by weight of the polyurethane urea solution according to the invention and 18% by weight aqueous aluminum chlorohydrate solution (corresponding to 4% by weight of aluminum chlorohydrate (ACH)+14% by weight of water) were mixed at room temperature with 14% by weight of ethanol. A homogeneous, transparent solution was obtained, which formed a transparent film directly when applied by spraying onto the skin at room temperature.

### Resistance of a Composition Comprising Active Antiperspirant Ingredients on the Skin (Rub-Off Test):

15% by weight of aluminum chlorohydrate (ACH) were mixed with 10% by weight of water, and 10% by weight of the polyurethane urea solution according to the invention, 65% by weight of ethanol and 0.2% by weight of red pigments at room temperature.

The experiment was repeated, but the 10% by weight of the polyurethane urea solution according to the invention was replaced by ethanol.

From each composition, 0.5 g was applied to an area of 2×2 cm on the dry skin of a test individual, and dried at room temperature for 10 minutes. A cotton pad was then rubbed for 1 time for 5 seconds over the area of application, and consideration was given to the remaining amount of pigment on the skin, and also the amount transferred to the cotton pad.

Without addition of the inventive solution, a significant quantity was rubbed off, whereas with the polyurethane urea solution of the invention there was much less visible abrasion on the cotton pad.

The amount of pigment remaining on the skin was investigated using a colorimeter (BYK GARDNER GmbH, Color-guide sphere, CAT No. 6835, Spectrum Scale 400 to 700 nm, Light path d/8° spex, Sample Mode: D65/10°, Light Source D65, Viewer angle: 10°, Calibration with white and black standard board), and a determination was made of the A value (high: high red component, low: high green component) before and after rubbing of the cotton pad. Before rubbing, both samples on the skin had an A value of 35. After rubbing, the skin in the sample region, in the case of the sample without the inventive polyurethane urea solution, had an A value of just 20, whereas the sample with the inventive solution still had an A value of 34.

The test was repeated under the same conditions, except that the cotton pad was impregnated with water at room temperature before rubbing took place. After 5 seconds of rubbing with a wet cotton pad as well, a red coloration on the cotton pad was clearly visible for the sample without the inventive solution, whereas, for the sample containing the inventive solution, only traces of red abrasion were obtained on the cotton pad.

The amount of pigment remaining on the skin was again investigated using a colorimeter, and a determination was made of the A value (high: high red component, low: high green component) before and after rubbing with the cotton pad. Before rubbing, the samples on the skin had an A value of 35 (without inventive solution) and 40 (with inventive solution). After rubbing, the skin in the sample area, in the case of the sample without the inventive polyurethane urea solution, only had an A value of 19, whereas the sample with the inventive solution still had an A value of 32.

The inventive solution therefore significantly increased the resistance on the skin of the compositions comprising active antiperspirant ingredient.

This specification has been written with reference to various non-limiting and non-exhaustive embodiments. However, it will be recognized by persons having ordinary skill in the art that various substitutions, modifications, or combinations of any of the disclosed embodiments (or portions thereof) may be made within the scope of this specification. Thus, it is contemplated and understood that this specification supports additional embodiments not expressly set forth herein. Such embodiments may be obtained, for example, by combining, modifying, or reorganizing any of the disclosed steps, components, elements, features, aspects, characteristics, limitations, and the like, of the various non-limiting embodiments described in this specification. In this manner, Applicant(s) reserve the right to amend the claims during prosecution to add features as variously described in this specification, and such amendments comply with the requirements of 35 U.S.C. § 112(a), and 35 U.S.C. § 132(a).

Various aspects of the subject matter described herein are set out in the following numbered clauses:
1. A composition for application to the human and animal body or to plants, comprising at least one active ingredient and/or fragrance, characterized in that it further comprises a polyurethane urea which is present dissolved in a solvent or solvent mixture, the solvent consisting exclusively of one or more monohydroxy-functional alcohols, or a solvent mixture consisting of organic solvents, which contains ≥50% by weight, based on the total mass of the solvent mixture, of at least one monohydroxy-functional alcohol, is used.
2. The composition according to clause 1, characterized in that the polyurethane urea is constructed from
   a) at least one aliphatic, araliphatic and/or cycloaliphatic diisocyanate,
   b) at least one polyetherpolyol having a number-average molecular weight Mn of ≥400 and ≤6000 g/mol and a hydroxyl functionality of ≥1.5 and ≤4,
   c) at least one amino-functional compound which has at least two isocyanate-reactive amino groups,
   d) optionally at least one alcohol which has at least two hydroxyl groups and a molar mass of ≥60 and ≤399 g/mol,
   e) optionally at least one compound which has one group that is reactive towards isocyanate groups, and
   f) optionally ≤20% by weight, based on the total mass of the polyurethane urea, of at least one polyol having a number-average molecular weight Mn of ≥500 and ≤6000 g/mol and a hydroxyl functionality of ≥1.5 and ≤4 and being different from b).
3. A composition comprising at least one active ingredient and/or fragrance and at least one polyurethane urea which is present dissolved in a solvent or solvent mixture, the solvent consisting of one or more monohydroxy-functional alcohols, or a solvent mixture consisting of exclusively organic solvents, which contains ≥50% by weight, based on the total mass of the solvent mixture, of at least one monohydroxy-functional alcohol, is used, characterized in that the polyurethane urea is constructed from
   a) at least one aliphatic, araliphatic and/or cycloaliphatic diisocyanate,
   b) at least one polyetherpolyol having a number-average molecular weight Mn of ≥400 and ≤6000 g/mol and a hydroxyl functionality of ≥1.5 and ≤4,
   c) at least one amino-functional compound which has at least two isocyanate-reactive amino groups,
   d) optionally at least one alcohol which has at least two hydroxyl groups and a molar mass of ≥60 and ≤399 g/mol,
   e) optionally at least one compound which has one group that is reactive towards isocyanate groups, and
   f) optionally ≤20% by weight, based on the total mass of the polyurethane urea, of at least one polyol having a number-average molecular weight Mn of ≥500 and ≤6000 g/mol and a hydroxyl functionality of ≥1.5 and ≤4 and being different from b).
4. The composition according to any of clauses 1 to 3, characterized in that the polyurethane urea has no hydrophilizing groups.
5. The composition according to any of clauses 2 to 4, characterized in that component b) is selected from poly(tetramethylene glycol) polyetherpolyols.
6. The composition according to any of clauses 2 to 5, characterized in that component a) is selected from aliphatic, araliphatic and/or cycloaliphatic diisocyanates which have at least one isocyanate group which is bonded to a secondary and/or tertiary C atom.
7. The composition according to any of clauses 1 to 6, characterized in that the monohydroxy-functional alcohols are selected from aliphatic alcohols having one to six carbon atoms.
8. The composition according to any of clauses 1 to 7, characterized in that the solvent mixture contains ≥95% by weight, based on the total mass of the solvent mixture, of at least one monohydroxy-functional alcohol.
9. The composition according to any of clauses 1 to 8, characterized in that it is free from toluene, dimethylacetamide, dimethylformamide, γ-butyrolactone, N-methyl-2-pyrrolidinone.
10. The composition according to any of clauses 1 to 9, characterized in that it contains ≥0.5 and ≤20% by weight of the polyurethane urea as substance, based on the total weight of the composition.
11. The composition according to any of clauses 1 to 10, characterized in that it contains ≥0.01 and ≤40% by weight of the active ingredient and/or fragrance, based on the total weight of the composition.
12. The composition according to any of clauses 1 to 11, characterized in that the active ingredient has ionic groups.
13. The composition according to any of clauses 1 to 12 for treating the human or animal body.
14. Use of the composition according to any of clauses 1 to 12 to coat substrates, the substrates not being the human or animal body.
15. Use of the composition according to any of clauses 1 to 12 for application to the human or animal body, the active ingredient not having a therapeutic effect.

1. A composition for application to one of a human, an animal body, and a plant, the composition comprising at least one active ingredient and a fragrance, wherein the composition comprises a polyurethane urea dissolved in a solvent or solvent mixture, the solvent consisting of one or more monohydroxy-functional alcohols, the solvent mixture consisting of organic solvents, which contain ≥50% by weight, based on the total mass of the solvent mixture, of at least one monohydroxy-functional alcohol.

2. The composition according to claim 1, wherein the polyurethane urea comprises:
a) at least one aliphatic, araliphatic and/or cycloaliphatic diisocyanate,
b) at least one polyetherpolyol having a number-average molecular weight Mn of ≥400 and ≤6000 g/mol and a hydroxyl functionality of ≥1.5 and ≤4,
c) at least one amino-functional compound which has at least two isocyanate-reactive amino groups,
d) optionally, at least one alcohol which has at least two hydroxyl groups and a molar mass of ≥60 and ≤399 g/mol,
e) optionally, at least one compound which has one group that is reactive towards isocyanate groups, and
f) optionally, ≤20% by weight, based on the total mass of the polyurethane urea, of at least one polyol having a number-average molecular weight Mn of ≥500 and ≤6000 g/mol and a hydroxyl functionality of ≥1.5 and ≤4 and being different from b).

3. A composition comprising at least one active ingredient and a fragrance and at least one polyurethane urea dissolved in a solvent or solvent mixture, the solvent consisting of one or more monohydroxy-functional alcohols, the solvent mixture consisting of organic solvents, which contain ≥50% by weight, based on the total mass of the solvent mixture, of at least one monohydroxy-functional alcohol,
wherein the polyurethane urea comprises:
a) at least one aliphatic, araliphatic and/or cycloaliphatic diisocyanate,
b) at least one polyetherpolyol having a number-average molecular weight Mn of ≥400 and ≤6000 g/mol and a hydroxyl functionality of ≥1.5 and ≤4,
c) at least one amino-functional compound which has at least two isocyanate-reactive amino groups,
d) optionally, at least one alcohol which has at least two hydroxyl groups and a molar mass of ≥60 and ≤399 g/mol,
e) optionally, at least one compound which has one group that is reactive towards isocyanate groups, and
f) optionally, ≤20% by weight, based on the total mass of the polyurethane urea, of at least one polyol having a number-average molecular weight Mn of ≥500 and ≤6000 g/mol and a hydroxyl functionality of ≥1.5 and ≤4 and being different from b).

4. The composition according to claim 1, wherein the polyurethane urea has no hydrophilizing groups.

5. A composition according to claim 2, wherein component b) is selected from poly(tetramethylene glycol) polyetherpolyols.

6. The composition according to claim 2, wherein component a) is selected from aliphatic, araliphatic and cycloaliphatic diisocyanates which have at least one isocyanate group which is bonded to a secondary and/or tertiary carbon atom.

7. The composition according to claim 1, wherein the monohydroxy-functional alcohols are selected from aliphatic alcohols having one to six carbon atoms.

8. The composition according to claim 1, characterized in that the solvent mixture contains ≥95% by weight, based on the total mass of the solvent mixture, of at least one monohydroxy-functional alcohol.

9. The composition according to claim 1, characterized in that it is free from toluene, dimethylacetamide, dimethylformamide, γ-butyrolactone, N-methyl-2-pyrrolidinone.

10. The composition according to claim 1, wherein that composition contains ≥0.5 and ≤20% by weight of the polyurethane urea as substance, based on the total weight of the composition.

11. The composition according to claim 1, wherein the composition contains ≥0.01 and ≤40% by weight of at least one of the active ingredient and fragrance, based on the total weight of the composition.

12. The composition according to claim 1, characterized in that the active ingredient has ionic groups.

13. A composition for treating one of a human and an animal body comprising the composition according to claim 1.

14. The composition according to claim 1 applied to a substrates, wherein the substrate is not a human or an animal body.

15. The composition according to claim 1 applied to one of a human and an animal body, wherein the active ingredient has no therapeutic effect.
